(19) United States
(12) Patent Application Publication
(10) Pub. No.: US 2024/0409615 A1
(43) Pub. Date: Dec. 12, 2024

(54) MULTI-VECTOR RECOMBINASE MEDIATED CASSETTE EXCHANGE

(71) Applicant: Genentech, Inc., South San Francisco, CA (US)

(72) Inventors: Chi Kin Domingos Ng, San Francisco, CA (US); Amy Shen, San Mateo, CA (US); Gavin Christian Barnard, South San Francisco, CA (US)

(73) Assignee: Genentech, Inc., South San Francisco, CA (US)

(21) Appl. No.: 18/752,501

(22) Filed: Jun. 24, 2024

Related U.S. Application Data
(63) Continuation of application No. PCT/US2022/053761, filed on Dec. 22, 2022.
(60) Provisional application No. 63/292,869, filed on Dec. 22, 2021.

(51) Int. Cl. C07K 16/00; C12N 15/85

(52) U.S. Cl. CPC C07K 16/00; C12N 15/85; C12N 2800/107

(57) ABSTRACT

The presently disclosed subject matter relates to multi-vector recombinase mediated cassette exchange approaches to achieve targeted integration of sequences of interest for the generation host cells expressing recombinant proteins, e.g., monoclonal antibodies, as well as compositions derived from the same, e.g., bispecific antibodies, and other complex format proteins, e.g., membrane protein complexes, and other difficult to express molecules.

### CROSS-REFERENCE TO RELATED APPLICATIONS

This application is a continuation of International Application No. PCT/US2022/053761, filed Dec. 22, 2022, which claims priority to U.S. Provisional Application Ser. No. 63/292,869, filed Dec. 22, 2021, the disclosure of each is hereby incorporated by reference in its entirety.

### SEQUENCE LISTING

The instant application contains a Sequence Listing which has been submitted electronically in XML format and is hereby incorporated by reference in its entirety. Said XML copy, created on Jun. 24, 2024, is named 00B206.1444.xml and is 984,076 bytes in size.

### TECHNICAL FIELD

The presently disclosed subject matter relates to cell line development employing multi-vector recombinase mediated cassette exchange approaches to achieve targeted integration of sequences of interest for the generation host cells expressing recombinant proteins, e.g., monoclonal antibodies, as well as compositions derived from the same, e.g., bispecific antibodies, and other complex format proteins, e.g., membrane protein complexes, and other difficult to express molecules.

### BACKGROUND

Due to the rapid advancement in cell biology and immunology, there has been an increasing demand to develop novel therapeutic recombinant proteins, e.g., monoclonal antibodies, bispecific antibodies, and complex format proteins, for a variety of diseases including cancer, cardiovascular diseases, and metabolic diseases. These biopharmaceutical candidates are commonly manufactured by commercial cell lines capable of expressing the proteins of interest. For example, Chinese hamster ovary (CHO) cells have been widely adapted to produce therapeutic monoclonal or bispecific antibodies as well as more complex format proteins in recent years.

Conventional strategies for developing commercial cell lines generally involve repeated efforts directed to integrating a nucleotide sequence encoding the polypeptide of interest, either randomly or at a specific (“targeted”) location, followed by the selection and isolation of cell lines producing that polypeptide. There is, however, a need in the art for new cell line development strategies that not only conserve resources but also generate cell lines exhibiting improved expression levels, product quality attributes, and production culture performance relative to conventional methodologies.

### SUMMARY OF THE INVENTION

The presently disclosed subject matter relates to multi-vector recombinase mediated cassette exchange (multi-vector RMCE) approaches to achieve targeted integration of sequences of interest for the generation host cells expressing recombinant proteins, e.g., monoclonal antibodies, as well as compositions derived from the same, e.g., bispecific antibodies, and other complex format proteins, e.g., membrane protein complexes, and other difficult to express molecules.

In certain embodiments, the present disclosure is directed to TI host cells comprising an exogenous nucleotide sequence integrated at an integration site that is within a sequence: a) at least about 90% homologous to all or part of nucleotides 41190-45269 of NW_006874047.1, all or part of nucleotides 63590-207911 of NW_006884592.1, all or part of nucleotides 253831-491909 of NW_006881296.1, all or part of nucleotides 69303-79768 of NW_003616412.1, all or part of nucleotides 293481-315265 of NW_003615063.1, all or part of nucleotides 2650443-2662054 of NW_006882936.1, or all or part of nucleotides 82214-97705 of NW_003615411.1; or b) at least about 90% homologous to all or part of nucleotides 45270-45490 of NW_006874047.1, all or part of nucleotides 207912-792374 of NW_006884592.1, all or part of nucleotides 491910-667813 of NW_006881296.1, all or part of nucleotides 79769-100059 of NW_003616412.1, all or part of nucleotides 315266-362442 of NW_003615063.1, all or part of nucleotides 2662055-2701768 of NW_006882936.1, or all or part of nucleotides 97706-105117 of NW_003615411.1; and wherein the exogenous nucleotide sequence comprises four or more incompatible recombination recognition sequences (RRSs). In certain embodiments, the RRSs are selected from the group consisting of a LoxP sequence, a LoxP L3 sequence, a LoxP 2L sequence, a LoxFas sequence, a Lox5 1 1 sequence, a Lox2272 sequence, a Lox2372 sequence, a Lox5 171 sequence, a Loxm2 sequence, a Lox71 sequence, a Lox66 sequence, a FRT sequence, a Bxbl attP sequence, a Bxbl attB sequence, a φC31 attP sequence, and a φC31 attB sequence. In certain embodiments, the RRSs are recognized by recombinases selected from the group consisting of Cre recombinase, FLP recombinase, Bxbl integrase, and a φC31 integrase. In certain embodiments, the exogenous nucleotide sequence comprises a selection marker located between the 5′-most RRS and the next RRS in the 3′ direction. In certain embodiments, the selection marker is selected from the group consisting of aminoglycoside phosphotransferase (APH), hygromycin phosphotransferase (HYG), neomycin, G418 APH), dihydrofolate reductase (DHFR), thymidine kinase (TK), glutamine synthetase (GS), asparagine synthetase, tryptophan synthetase (indole), histidinol dehydrogenase (histidinol D), blasticidin, bleomycin, phleomycin, chloramphenicol, Zeocin, and mycophenolic acid. In certain embodiments, the cells comprise a second selection marker, wherein the first and the second selection markers are different. In certain embodiments, the second selection marker is selected from the group consisting of aminoglycoside phosphotransferase (APH), hygromycin phosphotransferase (HYG), neomycin, G418 APH), dihydrofolate reductase (DHFR), thymidine kinase (TK), glutamine synthetase (GS), asparagine synthetase, tryptophan synthetase (indole), histidinol dehydrogenase (histidinol D), blasticidin, bleomycin, phleomycin, chloramphenicol, Zeocin, and mycophenolic acid. In certain embodiments, the cells comprise a third selection marker and an internal ribosome entry site (IRES), wherein the IRES is operably linked to a third selection marker. In certain embodiments, the third selection marker is different from the first or the second selection marker. In certain embodiments, the third selection marker is selected from the group consisting of a green fluorescent protein (GFP) marker, an enhanced GFP (eGFP) marker, a synthetic GFP marker, a yellow fluorescent protein (YFP) marker, an enhanced YFP (eYFP) marker, a cyan fluorescent protein (CFP) marker, a mPlum marker, a mCherry marker, a tdTomato marker, a mStrawberry marker, a J-red marker, a DsRed-monomer marker, a mOrange marker, a mKO marker, a mCitrine marker, a Venus marker, a YPet marker, an Emerald6 marker, a CyPet marker, a mCFPm marker, a Cerulean marker, and a T-Sapphire marker. In certain embodiments, the TI host cell is a mammalian host cell. In certain embodiments, the TI host cell is a hamster host cell, a human host cell, a rat host cell, or a mouse host cell. In certain embodiments, the TI host cell is a Chinese hamster ovary (CHO) host cell, a CHO K1 host cell, a CHO K1 SV host cell, a DG44 host cell, a DUKXB-11 host cell, a CHOK1 S host cell, or a CHO K1M host cell.

In certain embodiments, the present disclosure is directed to methods of preparing a TI host cell expressing SOIs comprising: a) providing a TI host cell comprising an exogenous nucleotide sequence integrated at an integration site that is within a sequence: (A) at least about 90% homologous to all or part of nucleotides 41190-45269 of NW_006874047.1, all or part of nucleotides 63590-207911 of NW_006884592.1, all or part of nucleotides 253831-491909 of NW_006881296.1, all or part of nucleotides 69303-79768 of NW_003616412.1, all or part of nucleotides 293481-315265 of NW_003615063.1, all or part of nucleotides 2650443-2662054 of NW_006882936.1, or all or part of nucleotides 82214-97705 of NW_003615411.1; or (B) at least about 90% homologous to all or part of nucleotides 45270-45490 of NW_006874047.1, all or part of nucleotides 207912-792374 of NW_006884592.1, all or part of nucleotides 491910-667813 of NW_006881296.1, all or part of nucleotides 79769-100059 of NW_003616412.1, all or part of nucleotides 315266-362442 of NW_003615063.1, all or part of nucleotides 2662055-2701768 of NW_006882936.1, or all or part of nucleotides 97706-105117 of NW_003615411.1; and wherein the exogenous nucleotide sequence comprises four or more incompatible RRSs; b) introducing into the cell provided in a) at least three vectors, each vector comprising: (A) two RRSs matching two sequentially oriented RRSs on the integrated exogenous nucleotide sequence; and (B) each pair of RRSs flanking at least one exogenous SOI and at least one second selection marker; c) introducing recombinases or nucleic acids encoding recombinases, wherein the recombinases recognize the RRSs; and d) selecting for TI cells expressing the selection markers to thereby isolate a TI host cell expressing the SOIs. In certain embodiments, the recombinases are selected from Cre recombinase, FLP recombinase, Bxbl integrase, and a φC31 integrase. In certain embodiments, the SOIs encode a single chain antibody, an antibody light chain, an antibody heavy chain, a single-chain Fv fragment (scFv), or an Fc fusion protein. In certain embodiments, the TI host cell is a mammalian host cell. In certain embodiments, the TI host cell is a hamster host cell, a human host cell, a rat host cell, or a mouse host cell. In certain embodiments, the TI host cell is a CHO host cell, a CHO K1 host cell, a CHO K1SV host cell, a DG44 host cell, a DUKXB-11 host cell, a CHOK1S host cell, or a CHO K1M host cell. In certain embodiments, the vector is selected from the group consisting of an adenovirus vector, an adeno-associated virus vector, a lentivirus vector, a retrovirus vector, an integrating phage vector, a non-viral vector, a transposon and/or transposase vector, an integrase substrate, and a plasmid.

In certain embodiments, the present disclosure is directed to methods for expressing SOIs comprising: a) providing a TI host cell comprising an exogenous nucleotide sequence integrated at an integration site that is within a sequence: (A) at least about 90% homologous to all or part of nucleotides 41190-45269 of NW_006874047.1, all or part of nucleotides 63590-207911 of NW_006884592.1, all or part of nucleotides 253831-491909 of NW_006881296.1, all or part of nucleotides 69303-79768 of NW_003616412.1, all or part of nucleotides 293481-315265 of NW_003615063.1, all or part of nucleotides 2650443-2662054 of NW_006882936.1, or all or part of nucleotides 82214-97705 of NW_003615411.1; or (B) at least about 90% homologous to all or part of nucleotides 45270-45490 of NW_006874047.1, all or part of nucleotides 207912-792374 of NW_006884592.1, all or part of nucleotides 491910-667813 of NW_006881296.1, all or part of nucleotides 79769-100059 of NW_003616412.1, all or part of nucleotides 315266-362442 of NW_003615063.1, all or part of nucleotides 2662055-2701768 of NW_006882936.1, or all or part of nucleotides 97706-105117 of NW_003615411.1; and wherein the exogenous nucleotide sequence comprises four or more incompatible RRSs; b) introducing into the cell provided in a) at least three vectors, each vector comprising: (A) two RRSs matching two sequentially oriented RRSs on the integrated exogenous nucleotide sequence; and (B) each pair of RRSs flanking at least one exogenous SOI and at least one second selection marker; c) introducing recombinases or nucleic acids encoding recombinases, wherein the recombinases recognize the RRSs; d) selecting for TI cells expressing the selection markers to thereby isolate a TI host cell expressing the SOIs; and e) culturing the cell in d) under conditions suitable for expressing the SOIs and recovering the expressed protein therefrom. In certain embodiments, the recombinases are selected from Cre recombinase, FLP recombinase, Bxbl integrase, and a φC31 integrase. In certain embodiments, the SOIs encode a single chain antibody, an antibody light chain, an antibody heavy chain, a single-chain Fv fragment (scFv), or an Fc fusion protein. In certain embodiments, the TI host cell is a mammalian host cell. In certain embodiments, the TI host cell is a hamster host cell, a human host cell, a rat host cell, or a mouse host cell. In certain embodiments, the TI host cell is a CHO host cell, a CHO K1 host cell, a CHO K1SV host cell, a DG44 host cell, a DUKXB-11 host cell, a CHOK1S host cell, or a CHO K1M host cell. In certain embodiments, the vector is selected from the group consisting of an adenovirus vector, an adeno-associated virus vector, a lentivirus vector, a retrovirus vector, an integrating phage vector, a non-viral vector, a transposon and/or transposase vector, an integrase substrate, and a plasmid.

In certain embodiments, the present disclosure is directed to methods for producing a recombinant mammalian cell comprising a nucleic acid encoding an antibody, comprising: a) providing a mammalian cell comprising at least a single exogenous nucleic acid incorporated at a predetermined locus of the genome of the mammalian cell comprising four or more incompatible RRSs; b) introducing into the recombinant mammalian cell of a), at least three vectors, each vector comprising a pair of incompatible RRSs matching two of the incompatible RRSs comprised in the exogenous nucleic acid incorporated at a predetermined locus of the genome of the mammalian cell and each pair of incompatible RRSs flank one or more SOIs where the SOIs encode an antibody and/or one or more selection markers; c) introducing one or more recombinases, simultaneously or sequentially, with the introduction of the at least three vectors comprising the SOIs and/or selection markers; and d) selecting for cells expressing one or more of the SOIs and/or selection markers, thereby producing a recombinant mammalian cell comprising nucleic acid SOIs encoding the antibody. In certain embodiments, the exogenous nucleic acid is incorporated at a locus: (A) at least about 90% homologous to all or part of nucleotides 41190-45269 of NW_006874047.1, all or part of nucleotides 63590-207911 of NW_006884592.1, all or part of nucleotides 253831-491909 of NW_006881296.1, all or part of nucleotides 69303-79768 of NW_003616412.1, all or part of nucleotides 293481-315265 of NW_003615063.1, all or part of nucleotides 2650443-2662054 of NW_006882936.1, or all or part of nucleotides 82214-97705 of NW_003615411.1; or (B) at least about 90% homologous to all or part of nucleotides 45270-45490 of NW_006874047.1, all or part of nucleotides 207912-792374 of NW_006884592.1, all or part of nucleotides 491910-667813 of NW_006881296.1, all or part of nucleotides 79769-100059 of NW_003616412.1, all or part of nucleotides 315266-362442 of NW_003615063.1, all or part of nucleotides 2662055-2701768 of NW_006882936.1, or all or part of nucleotides 97706-105117 of NW_003615411.1. In certain embodiments, the recombinases are selected from Cre recombinase, FLP recombinase, Bxbl integrase, and a φC31 integrase. In certain embodiments, the SOIs encode a single chain antibody, an antibody light chain, an antibody heavy chain, a single-chain Fv fragment (scFv), or an Fc fusion protein. In certain embodiments, the mammalian cell is a hamster host cell, a human host cell, a rat host cell, or a mouse host cell. In certain embodiments, the mammalian cell is a CHO host cell, a CHO K1 host cell, a CHO K1SV host cell, a DG44 host cell, a DUKXB-11 host cell, a CHOK1S host cell, or a CHO K1M host cell. In certain embodiments, the vectors are selected from the group consisting of adenovirus vectors, adeno-associated virus vectors, lentivirus vectors, retrovirus vectors, integrating phage vectors, non-viral vectors, transposon and/or transposase vectors, integrase substrates, and plasmids.

In certain embodiments, the present disclosure is directed to methods for producing a recombinant mammalian cell comprising nucleic acids encoding one or more antibodies, comprising: a) providing a mammalian cell comprising at least two exogenous nucleic acid incorporated at predetermined loci of the genome of the mammalian cell comprising, each exogenous nucleic acid comprising four or more incompatible RRSs, where the exogenous nucleic acids can comprise the same or different RRSs; b) introducing into the recombinant mammalian cell of a), at least three vectors, each vector comprising a pair of incompatible RRSs matching two of the incompatible RRSs comprised in one or both of the exogenous nucleic acids incorporated at predetermined loci of the genome of the mammalian cell and each pair of incompatible RRSs flank one or more SOIs where the SOIs encode an antibody and/or one or more selection markers; c) introducing one or more recombinases, simultaneously or sequentially, with the introduction of the at least three vectors comprising the SOIs and/or selection markers; and d) selecting for cells expressing one or more of the SOIs and/or selection markers, thereby producing a recombinant mammalian cell comprising nucleic acid SOIs encoding the antibody. In certain embodiments, the exogenous nucleic acids are incorporated at distinct loci selected from loci: (A) at least about 90% homologous to all or part of nucleotides 41190-45269 of NW_006874047.1, all or part of nucleotides 63590-207911 of NW_006884592.1, all or part of nucleotides 253831-491909 of NW_006881296.1, all or part of nucleotides 69303-79768 of NW_003616412.1, all or part of nucleotides 293481-315265 of NW_003615063.1, all or part of nucleotides 2650443-2662054 of NW_006882936.1, or all or part of nucleotides 82214-97705 of NW_003615411.1; or (B) at least about 90% homologous to all or part of nucleotides 45270-45490 of NW_006874047.1, all or part of nucleotides 207912-792374 of NW_006884592.1, all or part of nucleotides 491910-667813 of NW_006881296.1, all or part of nucleotides 79769-100059 of NW_003616412.1, all or part of nucleotides 315266-362442 of NW_003615063.1, all or part of nucleotides 2662055-2701768 of NW_006882936.1, or all or part of nucleotides 97706-105117 of NW_003615411.1. In certain embodiments, the recombinases are selected from Cre recombinase, FLP recombinase, Bxbl integrase, and a φC31 integrase. In certain embodiments, the SOIs encode a single chain antibody, an antibody light chain, an antibody heavy chain, a single-chain Fv fragment (scFv), or an Fc fusion protein. In certain embodiments, the mammalian cell is a hamster host cell, a human host cell, a rat host cell, or a mouse host cell. In certain embodiments, the mammalian cell is a CHO host cell, a CHO K1 host cell, a CHO K1SV host cell, a DG44 host cell, a DUKXB-11 host cell, a CHOK1S host cell, or a CHO K1M host cell. In certain embodiments, the vectors are selected from the group consisting of adenovirus vectors, adeno-associated virus vectors, lentivirus vectors, retrovirus vectors, integrating phage vectors, non-viral vectors, transposon and/or transposase vectors, integrase substrates, and plasmids.

### BRIEF DESCRIPTION OF THE DRAWINGS

FIG. 1 depicts an exemplary multi-vector RMCE strategy of the present disclosure.

FIG. 2 depicts an exemplary multi-vector RMCE strategy of the present disclosure.

FIG. 3 depicts an exemplary multi-vector RMCE strategy of the present disclosure.

FIG. 4 depicts an exemplary multi-vector RMCE strategy of the present disclosure.

FIG. 5 depicts an exemplary multi-vector RMCE strategy of the present disclosure.

FIG. 6 depicts the copy number results of the CHO cells modified by two-vector RMCE as compared to three-vector RMCE. The gene copy number of the CHO clones is similar to expectations, where 2 HC and 4 LC were expected for two-vector RMCE clones and 3 HC and 6 LC were expected for three-vector RMCE clones.

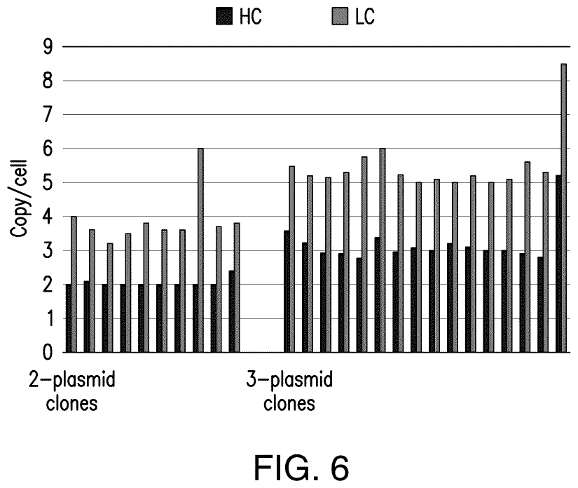

FIG. 7 depicts an exemplary multi-vector RMCE strategy of the present disclosure.

FIGS. 8A-8B depict comparisons of the productivities of TI transfection pools using two-plasmid based RMCEs and 2-site multi-vector plasmid (in this case, three plasmid) based RMCEs. Titer (bars) and Qp (dots) are both higher for the 2-site multi-vector plasmid based TI pools of mAb A (FIG. 8A) and TI pools of mAb B (FIG. 8b).

FIGS. 9A-9B graphically depicts certain CLD parameters assessed for the various TI pools of mAb C, mAb D, and mAb E, including titer (FIG. 9A) and Qp (FIG. 9B).

FIGS. 10A-10C graphically illustrates the titer observed for each eight mAb C clones in an Ambr15® culture (FIG. 10A) and the Qp observed for each of those mAb C eight clones (FIG. 10B), as well as well as provides a comparison of the average mAb C clone titers and mAb C clone Qp (FIG. 10C).

FIGS. 11A-11C graphically illustrates the titer observed for each eight mAb D clones in an Ambr15® culture (FIG. 11A) and the Qp observed for each of those mAb D eight clones (FIG. 11B), as well as well as provides a comparison of the average mAb D clone titers and mAb D clone Qp (FIG. 11C).

FIGS. 12A-12B compares titer (FIG. 12A) and Qp (FIG. 12B) for two specific clones (Clone A and Clone B) for a two vector approach and a multi-vector approach, respectively.

### DETAILED DESCRIPTION

The presently disclosed subject matter relates to multi-vector RMCE approaches to achieve targeted integration of sequences of interest for the generation host cells expressing recombinant proteins, e.g., monoclonal antibodies, as well as compositions derived from the same, e.g., bispecific antibodies, and other complex format proteins, e.g., membrane protein complexes, and other difficult to express molecules.

The presently disclosed subject matter offers particular benefits relative to conventional targeted integration-based approaches. For example, if three vectors, e.g., plasmids, are integrated simultaneously, then at least 15 genes (5 chains/donor plasmid) can be introduced at a single TI site simultaneously for titer improvement, improved product quality, increased cell fitness, etc. Similarly, if 4 plasmids are integrated simultaneously, at least 20 genes (5 chains/donor plasmid) for titer improvement, improved product quality, increased cell fitness, etc.

The multi-vector RMCE approaches described herein also allow for streamlined vector, e.g., plasmid, construction. In certain embodiments, such construction can be streamlined with a 1-step 6-way ligation for both mAb and 1-cell complex formats with up to 9 chains at a single integration site. Similarly, the multi-vector RMCE approaches described herein also allow for complex bispecific and trispecific antibody containing 4 or 5 or more unique genes to be integrated into the host genome simultaneously to generate cell lines expressing high titer and high product quality product. In addition, three vector RMCE allows for unique plasmids containing all the genes required to make gene therapy products can be integrated into the host genome simultaneously. Finally, additional unique RSS sites can be added via Crispr/Cas or Zinc finger or TALEN or random integration and if these unique RSS sites correspond to recombinases other than Cre and Flp (e.g. φC31 integrase & Bxbl and others), this will facilitate the simultaneous integration of additional genes in one or more landing pads

For purposes of clarity of disclosure and not by way of limitation, the detailed description is divided into the following subsections:
1. Definitions
2. Exogenous Nucleotide Sequences
3. Host Cells
4. Targeted Integration
5. Products

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the presently disclosed subject matter. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of”, and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “selection marker” can be a gene that allows cells carrying the gene to be specifically selected for or against, in the presence of a corresponding selection agent. For example, but not by way of limitation, a selection marker can allow the host cell transformed with the selection marker gene to be positively selected for in the presence of the gene; a non-transformed host cell would not be capable of growing or surviving under the selective conditions. Selection markers can be positive, negative or bi-functional. Positive selection markers can allow selection for cells carrying the marker, whereas negative selection markers can allow cells carrying the marker to be selectively eliminated. A selection marker can confer resistance to a drug or compensate for a metabolic or catabolic defect in the host cell. In prokaryotic cells, amongst others, genes conferring resistance against ampicillin, tetracycline, kanamycin or chloramphenicol can be used. Resistance genes useful as selection markers in eukaryotic cells include, but are not limited to, genes for aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid. Further marker genes are described in WO 92/08796 and WO 94/28143.

Beyond facilitating a selection in the presence of a corresponding selection agent, a selection marker can alternatively provide a gene encoding a molecule normally not present in the cell, e.g., green fluorescent protein (GFP), enhanced GFP (eGFP), synthetic GFP, yellow fluorescent protein (YFP), enhanced YFP (eYFP), cyan fluorescent protein (CFP), mPlum, mCherry, tdTomato, mStrawberry, J-red, DsRed-monomer, mOrange, mKO, mCitrine, Venus, YPet, Emerald, CyPet, mCFPm, Cerulean, and T-Sapphire. Cells harboring such a gene can be distinguished from cells not harboring this gene, e.g., by the detection of the fluorescence emitted by the encoded polypeptide.

As used herein, the term “operably linked” refers to a juxtaposition of two or more components, wherein the components are in a relationship permitting them to function in their intended manner. For example, a promoter and/or an enhancer is operably linked to a coding sequence if the promoter and/or enhancer acts to modulate the transcription of the coding sequence. In certain embodiments, DNA sequences that are “operably linked” are contiguous and adjacent on a single chromosome. In certain embodiments, e.g., when it is necessary to join two protein encoding regions, such as a secretory leader and a polypeptide, the sequences are contiguous, adjacent, and in the same reading frame. In certain embodiments, an operably linked promoter is located upstream of the coding sequence and can be adjacent to it. In certain embodiments, e.g., with respect to enhancer sequences modulating the expression of a coding sequence, the two components can be operably linked although not adjacent. An enhancer is operably linked to a coding sequence if the enhancer increases transcription of the coding sequence. Operably linked enhancers can be located upstream, within, or downstream of coding sequences and can be located a considerable distance from the promoter of the coding sequence. Operable linkage can be accomplished by recombinant methods known in the art, e.g., using PCR methodology and/or by ligation at convenient restriction sites. If convenient restriction sites do not exist, then synthetic oligonucleotide adaptors or linkers can be used in accord with conventional practice. An internal ribosomal entry site (IRES) is operably linked to an open reading frame (ORF) if it allows initiation of translation of the ORF at an internal location in a 5′ end-independent manner.

As used herein, the term “expression” refers to transcription and/or translation. In certain embodiments, the level of transcription of a desired product can be determined based on the amount of corresponding mRNA that is present. For example, mRNA transcribed from a sequence of interest can be quantitated by PCR or by Northern hybridization. In certain embodiments, protein encoded by a sequence of interest can be quantitated by various methods, e.g. by ELISA, by assaying for the biological activity of the protein, or by employing assays that are independent of such activity, such as Western blotting or radioimmunoassay, using antibodies that recognize and bind to the protein.

The term “sequence of interest” is used herein to refer to a polypeptide sequence (or, in certain instances, a nucleic acid encoding a polypeptide sequence), where expression of the polypeptide sequence is of interest. Such polypeptide sequences can, in certain embodiments, comprise a subunit of a multi-subunit protein complex. In certain embodiments, such polypeptide sequences can comprise fragments of such subunits. Such polypeptide sequences can, in certain embodiments, comprise an antibody sequence, e.g., an antibody heavy chain or light chain sequence. In certain embodiments, such polypeptide sequences can comprise fragments of such antibody sequences.

The term “antibody” is used herein in the broadest sense and encompasses various antibody structures, including but not limited to monoclonal antibodies, polyclonal antibodies, multispecific antibodies (e.g., bispecific antibodies), half antibodies, and antibody fragments so long as they exhibit a desired antigen-binding activity.

As used herein “standard antibodies” and “standard monoclonal antibodies” are antibodies or antibody fragments having a single binding specificity. In certain embodiments, the single binding specificity of standard antibodies is the result of the pairing of a heavy chain sequence, or fragment thereof, with a light chain sequence, or fragment thereof.

As used herein “Bispecific Antibodies” or “BsAbs” are antibodies that can simultaneously bind two distinct epitopes, e.g., two distinct epitopes on two distinct antigens or two distinct epitopes on a single antigen. BsAbs encompass numerous distinct structures, including those comprising paired variable heavy (V_{H}) and light (V_{L}) domains of two distinct parental monoclonal antibodies resulting in one “arm” (i.e., one paired V_{H} and V_{L}) of the BsAb having the binding specificity of the first parental antibody and a second “arm” of the BsAb having the binding specificity of the second parental antibody. BsAbs are a subset of multispecific antibodies, where multispecific antibodies comprise at least two binding specificities (i.e., BsAbs), but also include trispecific antibodies as well as antibodies having higher numbers of specificities.

As used herein, the term “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

As used herein, the term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three hypervariable regions (HVRs). See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind to a particular antigen may be isolated using a V_{H} or V_{L} domain from an antibody that binds the antigen to screen a library of complementary V_{L} or V_{H} domains, respectively. See, e.g., Portolano et al., J. Immunol. 150: 880-887 (1993); Clarkson et al., Nature 352:624-628 (1991).

As used herein, the term “vector” refers to a nucleic acid molecule capable of propagating another nucleic acid to which it is linked. The term includes the vector as a self-replicating nucleic acid structure as well as the vector incorporated into the genome of a host cell into which it has been introduced. In certain embodiments, vectors direct the expression of nucleic acids to which they are operatively linked. Such vectors are referred to herein as “expression vectors.”

As used herein, the term “homologous sequences” refers to sequences that share a significant sequence similarity as determined by an alignment of the sequences. For example, two sequences can be about 50%, 60%, 70%, 80%, 90%, 95%, 99%, or 99.9% homologous. The alignment is carried out by algorithms and computer programs including, but not limited to, BLAST, FASTA, and HMME, which compares sequences and calculates the statistical significance of matches based on factors such as sequence length, sequence identify and similarity, and the presence and length of sequence mismatches and gaps. Homologous sequences can refer to both DNA and protein sequences.

As used herein, the term “flanking” refers to that a first nucleotide sequence is located at either a 5′ or 3′ end, or both ends of a second nucleotide sequence. The flanking nucleotide sequence can be adjacent to or at a defined distance from the second nucleotide sequence. There is no specific limit of the length of a flanking nucleotide sequence. For example, a flanking sequence can be a few base pairs or a few thousand base pairs.

As used herein, the term “exogenous” indicates that a nucleotide sequence does not originate from a host cell and is introduced into a host cell by traditional DNA delivery methods, e.g., by transfection, electroporation, or transformation methods. The term “endogenous” refers to that a nucleotide sequence originates from a host cell. An “exogenous” nucleotide sequence can have an “endogenous” counterpart that is identical in base compositions, but where the “exogenous” sequence is introduced into the host cell, e.g., via recombinant DNA technology.

As used herein, an “integration site” comprises a nucleic acid sequence within a host cell genome into which an exogenous nucleotide sequence is inserted. In certain embodiments, an integration site is between two adjacent nucleotides on the host cell genome. In certain embodiments, an integration site includes a stretch of nucleotide sequences. In certain embodiments, the integration site is located within a specific locus of the genome of the TI host cell. In certain embodiments, the integration site is within an endogenous gene of the TI host cell.

As used herein, a “recombinase recognition sequence” (RRS) is a nucleotide sequence recognized by a recombinase and is necessary and sufficient for recombinase-mediated recombination events. A RRS can be used to define the position where a recombination event will occur in a nucleotide sequence. As used herein “incompatible” RRSs are RRSs that are recognized by distinct recombinases.

As used herein, the term “TI host cell” refers to a cell comprising a genomic locus or loci, i.e., integration site(s), for use in expressing a sequence of interest. In certain embodiments, integration of SOIs into the TI host cell is facilitated by the presence of an exogenous nucleotide sequence at one more integration site comprising four or more RRSs, e.g., four or more incompatible RRSs.

### 2. Exogenous Nucleotide Sequences

The presently disclosed subject matter provides host cells suitable for the integration of exogenous nucleotides sequences. In certain embodiments, the exogenous nucleotide sequences serve as integration sites for use in a multi-vector (i.e., three or more vector) RMCE strategy, e.g., by comprising four or more recombinase recognition sequences. In certain embodiments, the exogenous nucleotide sequences serve as integration sites for use in a multi-vector RMCE strategy comprising four or more incompatible recombinase recognition sequences. In certain embodiments an exogenous nucleotide sequences codes for a sequence of interest. Accordingly, in certain embodiments, a host cell will comprise one or more exogenous nucleotide sequences that will facilitate the targeted integration of one or more exogenous nucleotide sequences coding for one or more sequences of interest. In certain embodiments, a host cell comprising an exogenous nucleotide sequence integrated at an integration site on the genome of the host cell is referred to as a TI host cell. Exogenous nucleotide sequences coding for one or more sequences of interest can be then introduced into the TI host cell and integration can be targeted to the integration site. As outlined below, a TI host cell may comprise multiple integration sites defined by the presence of an exogenous nucleotide sequence comprising elements, e.g., recombinase recognition sequences, that facilitate the integration of an exogenous nucleotide sequence coding for one or more sequences of interest.

In certain embodiments, an integration site and/or the nucleotide sequences flanking the integration site can be identified experimentally. In certain embodiments, an integration site and/or the nucleotide sequences flanking the integration site can be identified by genome-wide screening approaches to isolate host cells that express, at a desirable level, a polypeptide of interest encoded by one or more SOIs integrated into one or more exogenous nucleotide sequences, where the exogenous sequences are themselves integrated into one or more loci in the genome of the host cell. In certain embodiments, an integration site and/or the nucleotide sequences flanking an integration site can be identified by genome-wide screening approaches following transposase-based cassette integration event. In certain embodiments, an integration site and/or the nucleotide sequences flanking an integration site can be identified by brute force random integration screening. In certain embodiments, an integration site and/or the nucleotide sequences flanking an integration site can be determined by conventional sequencing approaches such as target locus amplification (TLA) followed by next-generation sequencing (NGS) and whole-genome NGS. In certain embodiments, the location of an integration site on a chromosome can be determined by conventional cell biology approaches such as fluorescence in-situ hybridization (FISH) analysis.

In certain embodiments, a host cell comprises a first exogenous nucleotide sequence integrated at a first integration site within a specific first locus in the genome of the host cell and a second exogenous nucleotide sequence integrated at a second integration site within a specific second locus in the genome. In certain embodiments, a host cell comprises multiple exogenous nucleotide sequences integrated at multiple integration sites in the genome of the host cell.

### 2.1 Exogenous Sequence Comprising Four or More RRSs

In certain embodiments, an integrated exogenous nucleotide sequence comprises four or more RRSs, wherein the RRS can be recognized by a recombinase. In certain embodiments, an integrated exogenous nucleotide sequence comprises four or more incompatible RRSs. In certain embodiments, an integrated exogenous nucleotide sequence comprises four, five, six, seven, or eight or more RRSs. In certain embodiments, the RRS or RRSs can be selected from the group consisting of a LoxP sequence, a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, a Lox66 sequence, a FRT sequence, a Bxbl attP sequence, a Bxbl attB sequence, a φC31 attP sequence, and a φC31 attB sequence.

In certain embodiments, the integrated exogenous nucleotide sequence comprises at least one selection marker. In certain embodiments, the integrated exogenous nucleotide sequence comprises at least four RRSs and at least one selection marker. In certain embodiments, the integrated exogenous nucleotide sequence comprises four incompatible RRSs and at least one selection marker. In certain embodiments, a selection marker is located between the first RRS and the second RRS. In certain embodiments, two of the four RRSs flank at least one selection marker, i.e., a first RRS is located 5′ upstream and a second RRS is located 3′ downstream of the selection marker. In certain embodiments, a first RRS is adjacent to the 5′ end of the selection marker and a second RRS is adjacent to the 3′ end of the selection marker.

In certain embodiments, a selection marker is located between a first and a second RRS and the two flanking RRSs are different. In certain embodiments, the first flanking RRS is a LoxP L3 sequence and the second flanking RRS is a LoxP 2L sequence. In certain embodiments, a LoxP L3 sequence is located 5′ of the selection marker and a LoxP 2L sequence is located 3′ of the selection marker. In certain embodiments, the first flanking RRS is a wild-type FRT sequence and the second flanking RRS is a mutant FRT sequence. In certain embodiments, the first flanking RRS is a Bxbl attP sequence and the second flanking RRS is a Bxbl attB sequence. In certain embodiments, the first flanking RRS is a φC31 attP sequence and the second flanking RRS is a φC31 attB sequence. In certain embodiments, the two RRSs are positioned in the same orientation. In certain embodiments, the two RRSs are both in the forward or reverse orientation. In certain embodiments, the two RRSs are positioned in opposite orientation.

In certain embodiments, a selection marker can be an aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, or mycophenolic acid. In certain embodiments, a selection marker can be a GFP, an eGFP, a synthetic GFP, a YFP, an eYFP, a CFP, an mPlum, an mCherry, a tdTomato, an mStrawberry, a J-red, a DsRed-monomer, an mOrange, an mKO, an mCitrine, a Venus, a YPet, an Emerald, a CyPet, an mCFPm, a Cerulean, or a T-Sapphire marker.

In certain embodiments, the integrated exogenous nucleotide sequence comprises two selection markers flanked by at least two of the four or more RRSs, wherein a first selection marker is different from a second selection marker. In certain embodiments, the two selection markers are both selected from the group consisting of a glutamine synthetase selection marker, a thymidine kinase selection marker, a HYG selection marker, and a puromycin resistance selection marker. In certain embodiments, the integrated exogenous nucleotide sequence comprises a thymidine kinase selection marker and a HYG selection marker. In certain embodiments, the first selection maker is selected from the group consisting of an aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid, and the second selection maker is selected from the group consisting of a GFP, an eGFP, a synthetic GFP, a YFP, an eYFP, a CFP, an mPlum, an mCherry, a tdTomato, an mStrawberry, a J-red, a DsRed-monomer, an mOrange, an mKO, an mCitrine, a Venus, a YPet, an Emerald, a CyPet, an mCFPm, a Cerulean, and a T-Sapphire marker. In certain embodiments, the first selection marker is a glutamine synthetase selection marker and the second selection marker is a GFP marker. In certain embodiments, the two RRSs flanking both selection markers are the same. In certain embodiments, the two RRSs flanking both selection markers are different.

In certain embodiments, the selection marker is operably linked to a promoter sequence. In certain embodiments, the selection marker is operably linked to an SV40 promoter. In certain embodiments, the selection marker is operably linked to a Cytomegalovirus (CMV) promoter.

In certain embodiments, the integrated exogenous nucleotide sequence comprises at least one selection marker and an IRES, wherein the IRES is operably linked to the selection marker. In certain embodiments, the selection marker operably linked to the IRES is selected from the group consisting of a GFP, an eGFP, a synthetic GFP, a YFP, an eYFP, a CFP, an mPlum, an mCherry, a tdTomato, an mStrawberry, a J-red, a DsRed-monomer, an mOrange, an mKO, an mCitrine, a Venus, a YPet, an Emerald, a CyPet, an mCFPm, a Cerulean, and a T-Sapphire marker. In certain embodiments, the selection marker operably linked to the IRES is a GFP marker. In certain embodiments, the integrated exogenous nucleotide sequence comprises an IRES and two selection markers flanked by at least two of the four or more RRSs, wherein the IRES is operably linked to the second selection marker. In certain embodiments, the integrated exogenous nucleotide sequence comprises an IRES and three selection markers flanked by at least two of the at least four RRSs, wherein the IRES is operably linked to the third selection marker. In certain embodiments, the integrated exogenous nucleotide sequence comprises an IRES and three selection markers flanked by at least two of the at least four RRSs, wherein the IRES is operably linked to the third selection marker. In certain embodiments, the third selection marker is different from the first or the second selection marker. In certain embodiments, the integrated exogenous nucleotide sequence comprises a first selection marker operably linked to a promoter and a second selection marker operably linked to an IRES. In certain embodiments, the integrated exogenous nucleotide sequence comprises a glutamine synthetase selection marker operably linked to a SV40 promoter and a GFP selection marker operably linked to an IRES. In certain embodiments, the integrated exogenous nucleotide sequence comprises a thymidine kinase selection marker and a HYG selection marker operably linked to a CMV promoter and a GFP selection marker operably linked to an IRES.

In certain embodiments the exogenous nucleotide sequence serving as an integration site will be present at a site within a specific locus of the genome of a TI host cell. Exemplary TI host cells and strategies for the use of the same are described in detail in U.S. Patent Application Publication No. US20210002669, the contents of which are incorporated by reference in their entirety.

In certain embodiments employing targeted integration, the exogenous nucleotide sequence is integrated at a site within a specific locus of the genome of a TI host cell. In certain embodiments, the locus into which the exogenous nucleotide sequence is integrated is at least about 50%, at least about 60%, at least about 70%, at least about 80%, at least about 90%, at least about 95%, at least about 99%, or at least about 99.9% homologous to a sequence selected from Contigs NW_006874047.1, NW_006884592.1, NW_006881296.1, NW_003616412.1, NW_003615063.1, NW_006882936.1, and NW_003615411.1.

In certain embodiments, the nucleotide sequence immediately 5′ of the integrated exogenous sequence is selected from the group consisting of nucleotides 41190-45269 of NW_006874047.1, nucleotides 63590-207911 of NW_006884592.1, nucleotides 253831-491909 of NW_006881296.1, nucleotides 69303-79768 of NW_003616412.1, nucleotides 293481-315265 of NW_003615063.1, nucleotides 2650443-2662054 of NW_006882936.1, or nucleotides 82214-97705 of NW_003615411.1 and sequences at least 50% homologous thereto. In certain embodiments, the nucleotide sequence immediately 5′ of the integrated exogenous sequence are at least about 50%, at least about 60%, at least about 70%, at least about 80%, at least about 90%, at least about 95%, at least about 99%, or at least about 99.9% homologous to nucleotides 41190-45269 of NW_006874047.1, nucleotides 63590-207911 of NW_006884592.1, nucleotides 253831-491909 of NW_006881296.1, nucleotides 69303-79768 of NW_003616412.1, nucleotides 293481-315265 of NW_003615063.1, nucleotides 2650443-2662054 of NW_006882936.1, or nucleotides 82214-97705 of NW_003615411.1.

In certain embodiments, the nucleotide sequence immediately 3′ of the integrated exogenous sequence is selected from the group consisting of nucleotides 45270-45490 of NW 006874047.1, nucleotides 207912-792374 of NW 006884592.1, nucleotides 491910-667813 of NW_006881296.1, nucleotides 79769-100059 of NW_003616412.1, nucleotides 315266-362442 of NW_003615063.1, nucleotides 2662055-2701768 of NW_006882936.1, or nucleotides 97706-105117 of NW_003615411.1 and sequences at least 50% homologous thereto. In certain embodiments, the nucleotide sequence immediately 3′ of the integrated exogenous sequence is at least about 50%, at least about 60%, at least about 70%, at least about 80%, at least about 90%, at least about 95%, at least about 99%, or at least about 99.9% homologous to nucleotides 45270-45490 of NW_006874047.1, nucleotides 207912-792374 of NW 006884592.1, nucleotides 491910-667813 of NW 006881296.1, nucleotides 79769-100059 of NW_003616412.1, nucleotides 315266-362442 of NW_003615063.1, nucleotides 2662055-2701768 of NW_006882936.1, or nucleotides 97706-105117 of NW_003615411.1.

In certain embodiments, the integrated exogenous sequence is flanked 5′ by a nucleotide sequence selected from the group consisting of nucleotides 41190-45269 of NW_006874047.1, nucleotides 63590-207911 of NW_006884592.1, nucleotides 253831-491909 of NW_006881296.1, nucleotides 69303-79768 of NW_003616412.1, nucleotides 293481-315265 of NW_003615063.1, nucleotides 2650443-2662054 of NW_006882936.1, and nucleotides 82214-97705 of NW_003615411.1. and sequences at least 50% homologous thereto. In certain embodiments, the integrated exogenous sequence is flanked 3′ by a nucleotide sequence selected from the group consisting of nucleotides 45270-45490 of NW_006874047.1, nucleotides 207912-792374 of NW_006884592.1, nucleotides 491910-667813 of NW_006881296.1, nucleotides 79769-100059 of NW_003616412.1, nucleotides 315266-362442 of NW_003615063.1, nucleotides 2662055-2701768 of NW_006882936.1, and nucleotides 97706-105117 of NW_003615411.1 and sequences at least 50% homologous thereto. In certain embodiments, the nucleotide sequence flanking 5′ of the integrated exogenous nucleotide sequence is at least about 50%, at least about 60%, at least about 70%, at least about 80%, at least about 90%, at least about 95%, at least about 99%, or at least about 99.9% homologous to nucleotides 41190-45269 of NW_006874047.1, nucleotides 63590-207911 of NW_006884592.1, nucleotides 253831-491909 of NW_006881296.1, nucleotides 69303-79768 of NW_003616412.1, nucleotides 293481-315265 of NW_003615063.1, nucleotides 2650443-2662054 of NW_006882936.1, and nucleotides 82214-97705 of NW_003615411.1. In certain embodiments, the nucleotide sequence flanking 3′ of the integrated exogenous nucleotide sequence is at least about 50%, at least about 60%, at least about 70%, at least about 80%, at least about 90%, at least about 95%, at least about 99%, or at least about 99.9% homologous to nucleotides 45270-45490 of NW 006874047.1, nucleotides 207912-792374 of NW 006884592.1, nucleotides 491910-667813 of NW_006881296.1, nucleotides 79769-100059 of NW_003616412.1, nucleotides 315266-362442 of NW_003615063.1, nucleotides 2662055-2701768 of NW_006882936.1, and nucleotides 97706-105117 of NW_003615411.1.

In certain embodiments, the integrated exogenous nucleotide sequence is operably linked to a nucleotide sequence selected from the group consisting of Contigs NW_006874047.1, NW_006884592.1, NW_006881296.1, NW_003616412.1, NW_003615063.1, NW_006882936.1, and NW_003615411.1 and sequences at least 50% homologous thereto. In certain embodiments, the nucleotide sequence operably linked to the exogenous nucleotide sequence is at least about 50%, at least about 60%, at least about 70%, at least about 80%, at least about 90%, at least about 95%, at least about 99%, or at least about 99.9% homologous to a sequence selected from Contigs NW_006874047.1, NW_006884592.1, NW_006881296.1, NW_003616412.1, NW_003615063.1, NW_006882936.1, and NW_003615411.1.

In certain embodiments, the nucleic acid encoding a product of interest can be integrated into a host cell genome using transposase-based integration. Transposase-based integration techniques are disclosed, for example, in Trubitsyna et al., Nucleic Acids Res. 45(10):e89 (2017), Li et al., PNAS 110(25):E2279-E2287 (2013) and WO 2004/009792, which are incorporated by reference herein in their entireties.

Table 1 provides exemplary TI host cell integration sites:

TABLE 1
TI host cell integration sites

| | | Contig | Integration | |
|---|---|---|---|---|
| Host | Contig | Size (kb) | site (bp) | Gene (SEQ ID No.) |
| 1 | NW_006874047.1 | 727 | 45269 | LOC107977062 |
| | | | | (SEQ ID No. 1) |
| 2 | NW_006884592.1 | 931 | 207911 | LOC100768845 |
| | | | | (SEQ ID No. 2) |
| 3 | NW_006881296.1 | 1016 | 491909 | ITPR2 (SEQ ID No. |
| | | | | 3) |
| 4 | NW_003616412.1 | 127 | 79768 | ERE67000.1 (SEQ |
| | | | | ID No. 4) |
| 5 | NW_003615063.1 | 372 | 315265 | UBAP2 (SEQ ID |
| | | | | No. 5) |
| 6 | NW_006882936.1 | 3042 | 2662054 | MTMR2 (SEQ ID |
| | | | | No. 6) |
| 7 | NW_003615411.1 | 277 | 97706 | XP_003512331.2 |
| | | | | (SEQ ID No. 7) |

### 2.2 Exogenous Nucleotide Sequence Comprising an SOI

In certain embodiments, the integrated exogenous nucleotide sequence comprises at least one exogenous SOI. In certain embodiments, the integrated exogenous nucleotide sequence comprises at least one selection marker and at least one exogenous SOI. In certain embodiments, the integrated exogenous nucleotide sequence comprises at least one selection marker, at least one exogenous SOI, and at least one RRS. In certain embodiments, the integrated exogenous nucleotide sequence comprises at least one, at least two, at least three, at least four, at least five, at least six, at least seven, at least eight, at least nine, at least ten, or more SOIs. In certain embodiments the SOIs are the same. In certain embodiments, the SOIs are different.

As noted above, in certain embodiments, the SOI encodes one or more subunits of a multi-subunit protein complex. In certain embodiments, such polypeptide sequences can comprise fragments of such subunit sequences. In certain embodiments, the sequences of interest can comprise combinations of such subunit sequences. For example, but not by way of limitation, such combinations can comprise one, two, three, four, five, six, seven, eight, nine, ten, or more of a first subunit sequence and/or one, two, three, four, five, six, seven, eight, nine, ten, or more sequences of a second, third, fourth, fifth, sixth, seventh, eighth, ninth, tenth, or more subunit sequence. Moreover, in certain embodiments, such combinations can comprise one, two, three, four, five, six, seven, eight, nine, ten, or more distinct variations of a first subunit sequence and/or one, two, three, four, five, six, seven, eight, nine, ten, or more distinct variations of a second, third, fourth, fifth, sixth, seventh, eighth, ninth, tenth, or more subunit sequence.

In certain embodiments the SOI encodes a single chain antibody or fragment thereof. In certain embodiments, the SOI encodes an antibody heavy chain sequence or fragment thereof. In certain embodiments, the SOI encodes an antibody light chain sequence or fragment thereof. In certain embodiments, an integrated exogenous nucleotide sequence comprises an SOI encoding an antibody heavy chain sequence or fragment thereof and an SOI encoding an antibody light chain sequence or fragment thereof. In certain embodiments, an integrated exogenous nucleotide sequence comprises an SOI encoding a first antibody heavy chain sequence or fragment thereof, an SOI encoding a second antibody heavy chain sequence or fragment thereof, and an SOI encoding an antibody light chain sequence or fragment thereof. In certain embodiments, an integrated exogenous nucleotide sequence comprises an SOI encoding a first antibody heavy chain sequence or fragment thereof, an SOI encoding a second antibody heavy chain sequence or fragment thereof, an SOI encoding a first antibody light chain sequence or fragment thereof and a second SOI encoding an antibody light chain sequence or fragment thereof. In certain embodiments, the number of SOIs encoding for heavy and light chain sequences can be selected to achieve a desired expression level of the heavy and light chain polypeptides, e.g., to achieve a desired amount of bispecific antibody production. In certain embodiments, the individual SOIs encoding heavy and light chain sequences can be integrated, e.g., into a single exogenous nucleic acid sequence present at a single integration site, into multiple exogenous nucleic acid sequences present at a single integration site, or into multiple exogenous nucleic acid sequences integrated at distinct integrations sites within the TI host cell.

In certain embodiments, the integrated exogenous nucleotide sequence comprises at least one selection marker, at least one exogenous SOI, and one RRS. In certain embodiments, the RRS is located adjacent to at least one selection marker or at least one exogenous SOI. In certain embodiments, the integrated exogenous nucleotide sequence comprises at least one selection marker, at least one exogenous SOI, and two RRSs. In certain embodiments, the integrated exogenous nucleotide sequence comprises at least one selection marker and at least one exogenous SOI located between the first and the second RRS. In certain embodiments, the two RRSs flanking the selection marker and the exogenous SOI are the same. In certain embodiments, the two RRSs flanking the selection marker and the exogenous SOI are different. In certain embodiments, the first flanking RRS is a LoxP L3 sequence and the second flanking RRS is a LoxP 2L sequence. In certain embodiments, a L3 LoxP sequence is located 5′ of the selection marker and the exogenous SOI, and a LoxP 2L sequence is located 3′ of the selection marker and the exogenous SOI.

In certain embodiments, the integrated exogenous nucleotide sequence comprises three RRSs and two exogenous SOIs, and the third RRS is located between the first and the second RRS. In certain embodiments, the first SOI is located between the first and the third RRS, and the second SOI is located between the third and the second RRS. In certain embodiments, the first and the second SOI are different. In certain embodiments, the first and the second RRS are the same and the third RRS is different from the first or the second RRS. In certain embodiments, all three RRSs are different. In certain embodiments, the first RRS is a LoxP L3 site, the second RRS is a LoxP 2L site, and the third RRS is a LoxFas site. In certain embodiments, the integrated exogenous nucleotide sequence comprises three RRSs, one exogenous SOI, and one selection marker. In certain embodiments, the SOI is located between the first and the third RRS, and the selection marker is located between the third and the second RRS. In certain embodiments, the integrated exogenous nucleotide sequence comprises three RRSs, two exogenous SOIs, and one selection marker. In certain embodiments, the first SOI and the selection marker are located between the first and the third RRS, and the second SOI is located between the third and the second RRS.

In certain embodiments, the exogenous SOI encodes a polypeptide of interest including, but not limited to, an antibody, an enzyme, a cytokine, a growth factor, a hormone, a viral protein, a bacterial protein, a vaccine protein, or a protein with therapeutic function. In certain embodiments, the exogenous SOI encodes an antibody or an antigen-binding fragment thereof. In certain embodiments, the exogenous SOI encodes a single chain antibody, an antibody light chain, an antibody heavy chain, a single-chain Fv fragment (scFv), or an Fc fusion protein. In certain embodiments, the exogenous SOI is operably linked to at least one cis-acting element, for example, a promoter or an enhancer. In certain embodiments, the exogenous SOI is operably linked to a CMV promoter.

In certain embodiments, the integrated exogenous nucleotide sequence comprises two RRSs and at least two exogenous SOIs located between the two RRSs. In certain embodiments, SOIs encoding one heavy chain and one light chain of an antibody are located between the two RRSs. In certain embodiments, SOIs encoding one heavy chain and two light chains of an antibody are located between the two RRSs. In certain embodiments, SOIs encoding different combinations of copies of heavy chain and light chain of an antibody are located between the two RRSs.

In certain embodiments, the integrated exogenous nucleotide sequence comprises three RRSs and at least two exogenous SOIs, and the third RRS is located between the first and the second RRS. In certain embodiments, at least one SOI is located between the first and the third RRS, and at least one SOI is located between the third and the second RRS. In certain embodiments, the first and the second RRS are the same and the third RRS is different from the first or the second RRS. In certain embodiments, all three RRSs are different. In certain embodiments, SOIs encoding one heavy chain and one light chain of a first antibody are located between the first and the third RRS, and SOIs encoding one heavy chain and one light chain of a second antibody are located between the third and the second RRS. In certain embodiments, SOIs encoding one heavy chain and two light chains of a first antibody are located between the first and the third RRS, and SOIs encoding one heavy chain and one light chain of a second antibody are located between the third RRS and the second RRS. In certain embodiments, SOIs encoding one heavy chain and three light chains of a first antibody are located between the first and the third RRS, and SOIs encoding one light chain of the first antibody and one heavy chain and one light chain of a second antibody are located between the third RRS and the second RRS. In certain embodiments, SOIs encoding one heavy chain and three light chains of a first antibody are located between the first and the third RRS, and SOIs encoding two light chains of the first antibody and one heavy chain and one light chain of a second antibody are located between the third RRS and the second RRS. In certain embodiments, SOIs encoding different combinations of copies of heavy chains and light chains of multiple antibodies are located between the first and the third RRS, and between the third and the second RRS.

### 3. Host Cells

In certain embodiments, a host cell is a eukaryotic host cell. In certain embodiments, a host cell is a mammalian host cell. In certain embodiments, a host cell is a hamster host cell, a human host cell, a rat host cell, or a mouse host cell. In certain embodiments, a host cell is a Chinese hamster ovary (CHO) host cell, a CHO K1 host cell, a CHO K1SV host cell, a DG44 host cell, a DUKXB-11 host cell, a CHOK1S host cell, or a CHO K1M host cell.

In certain embodiments, a host cell is selected from the group consisting of monkey kidney CV1 line transformed by SV40 (COS-7), human embryonic kidney line (293 or 293 cells as described, e.g., in Graham et al., J Gen Virol. 36:59 (1977)), baby hamster kidney cells (BHK), mouse sertoli cells (TM4 cells as described, e.g., in Mather, Biol. Reprod. 23:243-251 (1980)), monkey kidney cells (CV1), African green monkey kidney cells (VERO-76), human cervical carcinoma cells (HELA), canine kidney cells (MDCK; buffalo rat liver cells (BRL 3A), human lung cells (W138), human liver cells (Hep G2), mouse mammary tumor (MMT 060562), TRI cells, as described, e.g., in Mather et al., Annals N.Y. Acad. Sci. 383:44-68 (1982), MRC 5 cells, FS4 cells, Y0 cells, NS0 cells, Sp2/0 cells, and PER.C6® cells.

In certain embodiments, a host cell is a cell line. In certain embodiments, a host cell is a cell line that has been cultured for a certain number of generations. In certain embodiments, a host cell is a primary cell.

In certain embodiments, expression of a polypeptide of interest is stable if the expression level is maintained at certain levels, increases, or decreases less than 20%, over 10, 20, 30, 50, 100, 200, or 300 generations. In certain embodiments, expression of a polypeptide of interest is stable if the culture can be maintained without any selection. In certain embodiments, expression of a polypeptide of interest is high if the polypeptide product of the gene of interest reaches about 1 g/L, about 2 g/L, about 3 g/L, about 4 g/L, about 5 g/L, about 10 g/L, about 12 g/L, about 14 g/L, or about 16 g/L.

In certain embodiments, polypeptide of interest is produced and secreted into the cell culture medium. In certain embodiments, polypeptide of interest is expressed and retained within the host cell. In certain embodiments, polypeptide of interest is expressed, inserted into, and retained in the host cell membrane.

Exogenous nucleotides of interest or vectors can be introduced into a host cell by conventional cell biology methods including, but not limited to, transfection, transduction, electroporation, or injection. In certain embodiments, exogenous nucleotides of interest or vectors are introduced into a host cell by chemical-based transfection methods comprising lipid-based transfection method, calcium phosphate-based transfection method, cationic polymer-based transfection method, or nanoparticle-based transfection. In certain embodiments, exogenous nucleotides of interest are introduced into a host cell by virus-mediated transduction including, but not limited to, lentivirus, retrovirus, adenovirus, or adeno-associated virus-mediated transduction. In certain embodiments, exogenous nucleotides of interest or vectors are introduced into a host cell via gene gun-mediated injection. In certain embodiments, both DNA and RNA molecules are introduced into a host cell using methods described herein.

### 4. Targeted Integration

A targeted integration approach allows for exogenous nucleotide sequences to be integrated into one or more pre-determined sites of a host cell genome. In certain embodiments, the targeted integration is mediated by a recombinase that recognizes one or more RRSs. In certain embodiments, the targeted integration is mediated by homologous recombination.

### 4.1. Targeted Integration Via Multi-Vector RMCE

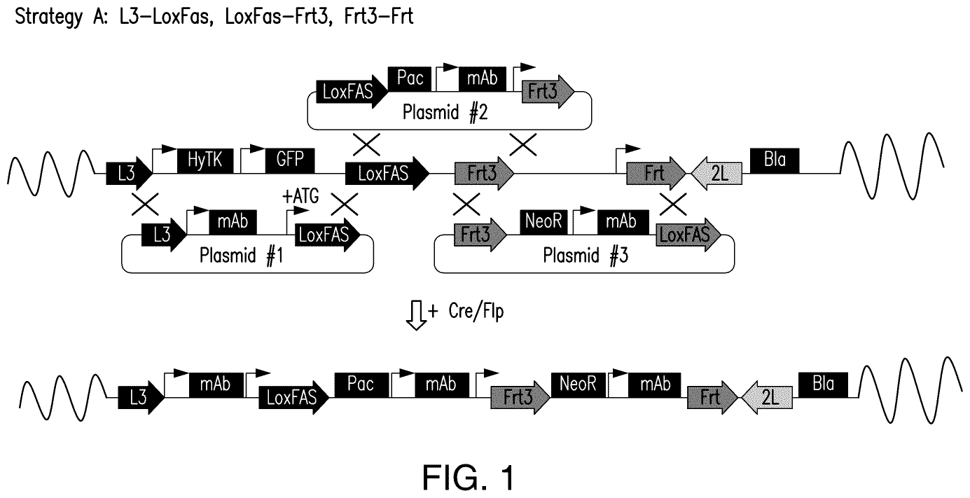

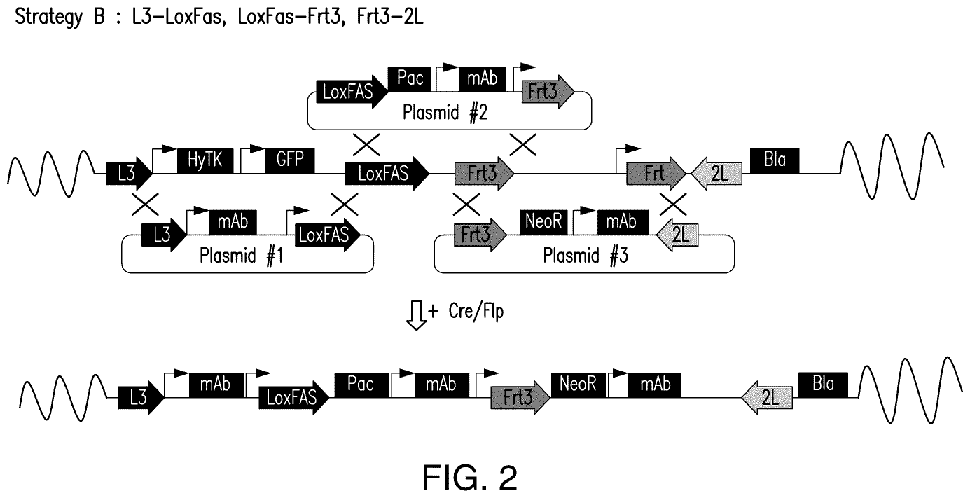

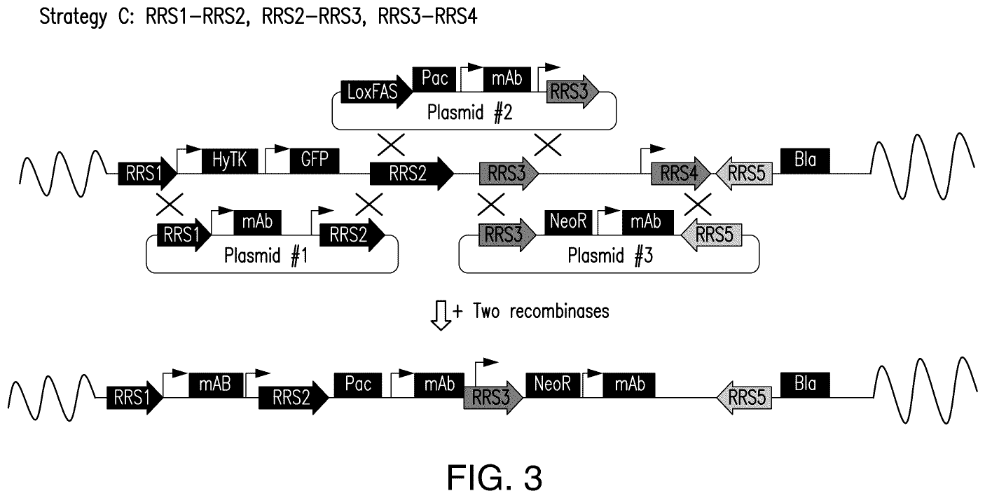

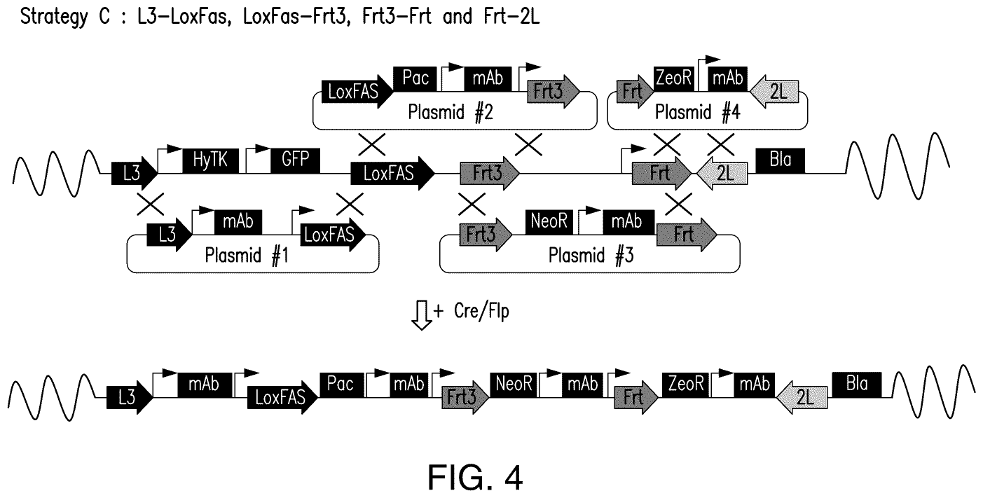

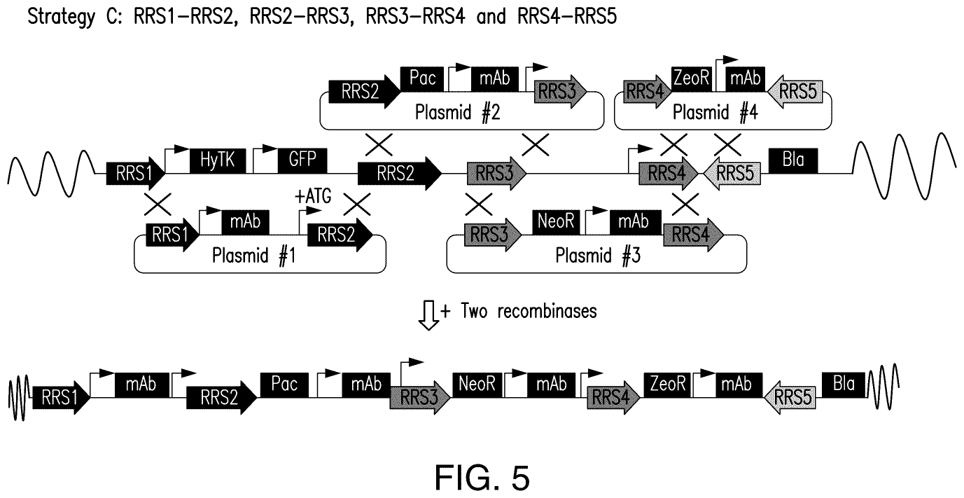

Multi-vector RMCE allows for unidirectional integration of one or more donor DNA molecule(s) into a pre-determined site of a host cell genome, and precise exchange of a DNA cassette present on the donor DNA with a DNA cassette on the host genome where the integration site resides. The DNA cassettes are characterized by four heterospecific RRSs, with at least two flanking at least one selection marker (although in certain RMCE examples a “split selection marker” can be used) and at least one exogenous SOI. RMCE involves double recombination cross-over events, catalyzed by a recombinase, between the two heterospecific RRSs within the target genomic locus and the donor DNA molecule. RMCE is designed to introduce a copy of the SOI or selection marker into the pre-determined locus of a host cell genome. Unlike recombination which involves just one cross-over event, RMCE can be implemented such that prokaryotic vector sequences are not introduced into the host cell genome, thus reducing and/or preventing unwanted triggering of host immune or defense mechanisms. The RMCE procedure can be repeated with multiple DNA cassettes. For example, the RMCE procedure can be employed to facilitate the introduction of multiple distinct “front” cassettes and multiple distinct “back” cassettes. As noted above, however, RMCE (and other integration strategies) can be employed to introduce as few as one cassette and as many as ten or more cassettes into a single pre-determined site of a host cell genome. For example, as outline in FIGS. 1-5, all three vectors can be used in a multi-vector RMCE approach to introduce three or more SOIs into a single locus. In certain embodiments, multi-RMCE can be used to introduce as few as one cassette and as many as ten or more cassettes simultaneously or sequentially into two, three, four, five, six, seven, or more distinct loci.

In certain embodiments, a RRS is selected from the group consisting of a LoxP sequence, a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, a Lox66 sequence, a FRT sequence, a Bxbl attP sequence, a Bxbl attB sequence, a φC31 attP sequence, and a φC31 attB sequence.

In certain embodiments, a RRS can be recognized by a Cre recombinase. In certain embodiments, a RRS can be recognized by a FLP recombinase. In certain embodiments, a RRS can be recognized by a Bxbl integrase. In certain embodiments, a RRS can be recognized by a φC31 integrase.

In certain embodiments when the RRS is a LoxP site, the host cell requires the Cre recombinase to perform the recombination. In certain embodiments when the RRS is a FRT site, the host cell requires the FLP recombinase to perform the recombination. In certain embodiments when the RRS is a Bxbl attP or a Bxbl attB site, the host cell requires the Bxbl integrase to perform the recombination. In certain embodiments when the RRS is a φC31 attP or a φC31attB site, the host cell requires the φC31 integrase to perform the recombination. The recombinases can be introduced into a host cell using an expression vector comprising coding sequences of the enzymes.

The Cre-LoxP site-specific recombination system has been widely used in many biological experimental systems. Cre is a 38-kDa site-specific DNA recombinase that recognizes 34 bp LoxP sequences. Cre is derived from bacteriophase P1 and belongs to the tyrosine family site-specific recombinase. Cre recombinase can mediate both intra and intermolecular recombination between LoxP sequences. The LoxP sequence is composed of an 8 bp nonpalindromic core region flanked by two 13 bp inverted repeats.

Cre recombinase binds to the 13 bp repeat thereby mediating recombination within the 8 bp core region. Cre-LoxP-mediated recombination occurs at a high efficiency and does not require any other host factors. If two LoxP sequences are placed in the same orientation on the same nucleotide sequence, Cre-mediated recombination will excise DNA sequences located between the two LoxP sequences as a covalently closed circle. If two LoxP sequences are placed in an inverted position on the same nucleotide sequence, Cre-mediated recombination will invert the orientation of the DNA sequences located between the two sequences. LoxP sequences can also be placed on different chromosomes to facilitate recombination between different chromosomes. If two LoxP sequences are on two different DNA molecules and if one DNA molecule is circular, Cre-mediated recombination will result in integration of the circular DNA sequence.

In certain embodiments, a LoxP sequence is a wild-type LoxP sequence. In certain embodiments, a LoxP sequence is a mutant LoxP sequence. Mutant LoxP sequences have been developed to increase the efficiency of Cre-mediated integration or replacement. In certain embodiments, a mutant LoxP sequence is selected from the group consisting of a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, and a Lox66 sequence. For example, the Lox71 sequence has 5 bp mutated in the left 13 bp repeat. The Lox66 sequence has 5 bp mutated in the right 13 bp repeat. Both the wild-type and the mutant LoxP sequences can mediate Cre-dependent recombination.

The FLP-FRT site-specific recombination system is similar to the Cre-Lox system. It involves the flippase (FLP) recombinase, which is derived from the 2 μm plasmid of the yeast Saccharomyces cerevisiae. FLP also belongs to the tyrosine family site-specific recombinase. The FRT sequence is a 34 bp sequence that consists of two palindromic sequences of 13 bp each flanking an 8 bp spacer. FLP binds to the 13 bp palindromic sequences and mediates DNA break, exchange and ligation within the 8 bp spacer. Similar to the Cre recombinase, the position and orientation of the two FRT sequences determine the outcome of FLP-mediated recombination. In certain embodiments, a FRT sequence is a wild-type FRT sequence. In certain embodiments, a FRT sequence is a mutant FRT sequence. Both the wild-type and the mutant FRT sequences can mediate FLP-dependent recombination. In certain embodiments, a FRT sequence is fused to a responsive receptor domain sequence, such as, but not limited to, a tamoxifen responsive receptor domain sequence.

Bxbl and φC31 belong to the serine recombinase family. They are both derived from bacteriophages and are used by these bacteriophages to establish lysogeny to facilitate site-specific integration of the phage genome into the bacterial genome. These integrases catalyze site-specific recombination events between short (40-60 bp) DNA substrates termed attP and attB sequences that are originally attachment sites located on the phage DNA and bacterial DNA, respectively. After recombination, two new sequences are formed, which are termed attL and attR sequences and each contains half sequences derived from attP and attB. Recombination can also occur between attL and attR sequences to excise the integrated phage out of the bacterial DNA. Both integrases can catalyze the recombination without the aid of any additional host factors. In the absence of any accessory factors, these integrases mediate unidirectional recombination between attP and attB with greater than 80% efficiency. Because of the short DNA sequences that can be recognized by these integrases and the unidirectional recombination, these recombination systems have been developed as a complement to the widely-used Cre-LoxP and FRT-FLP systems for genetic engineering purposes.

The term “matching RRSs” indicates that a recombination occurs between two RRSs. In certain embodiments, the two matching RRSs are the same. In certain embodiments, both RRSs are wild-type LoxP sequences. In certain embodiments, both RRSs are mutant LoxP sequences. In certain embodiments, both RRSs are wild-type FRT sequences. In certain embodiments, both RRSs are mutant FRT sequences. In certain embodiments, the two matching RRSs are different sequences but can be recognized by the same recombinase. In certain embodiments, the first matching RRS is a Bxbl attP sequence and the second matching RRS is a Bxbl attB sequence. In certain embodiments, the first matching RRS is a φC31 attB sequence and the second matching RRS is a φC31 attB sequence.

In certain embodiments, targeted integration is achieved by multiple RMCEs, wherein DNA cassettes from multiple vectors, each comprising at least an exogenous SOI or at least one selection marker flanked by two heterospecific RRSs, are all integrated into a pre-determined site of a host cell genome. In certain embodiments the selection marker can be partially encoded on the first the vector and partially encoded on the second, third, or subsequent vector such that the integration of multiple RMCEs allows for the expression of the selection marker.

In certain embodiments, targeted integration via recombinase-mediated recombination leads to a selection marker or one or more exogenous SOI integrated into one or more pre-determined integration sites of a host cell genome along with sequences from a prokaryotic vector. In certain embodiments, targeted integration via recombinase-mediated recombination leads to selection marker or one or more exogenous SOI integrated into one or more pre-determined integration sites of a host cell genome free of sequences from a prokaryotic vector. In certain embodiments of multi-vector RMCE, a selection marker and two SOIs can be incorporated into a first locus while an additional section marker and an addition SOI is incorporated into a second locus. Additionally, or alternatively, the methods described herein provide for the incorporation of a plurality of SOIs at a plurality of loci, where each locus can be the site of incorporation for one or more of SOIs.

In certain embodiments the sequence of interest comprises the sequence of one or more subunits of a multi-subunit protein complex. In certain embodiments, such polypeptide sequences can comprise fragments of such subunit sequences. In certain embodiments, the sequences of interest can comprise combinations of such subunit sequences. For example, but not by way of limitation, such combinations can comprise one, two, three, four, five, six, seven, eight, nine, ten, or more of a first subunit sequence and/or one, two, three, four, five, six, seven, eight, nine, ten, or more sequences of a second, third, fourth, fifth, sixth, seventh, eighth, ninth, tenth, or more subunit sequence. Moreover, in certain embodiments, such combinations can comprise one, two, three, four, five, six, seven, eight, nine, ten, or more distinct variations of a first subunit sequence and/or one, two, three, four, five, six, seven, eight, nine, ten, or more distinct variations of a second, third, fourth, fifth, sixth, seventh, eighth, ninth, tenth, or more subunit sequence.

In certain embodiments, the plurality of sequences of interest comprise one or more antibody heavy chain sequences (“H”) and/or one or more antibody light chain sequences (“L”). As used herein, such “H” and “L” sequences can be full length heavy or light chain sequences as well as heavy or light chain fragments, including, but not limited to, variable region fragments and complementary determining region fragments. In certain embodiments, the sequences of interest can comprise combinations of such H and L sequences. For example, but not by way of limitation, such combinations can comprise one, two, three, four, five, six, seven, eight, nine, ten, or more H sequences and/or one, two, three, four, five, six, seven, eight, nine, ten, or more L sequences. Moreover, in certain embodiments, such combinations can comprise one, two, three, four, five, six, seven, eight, nine, ten, or more of the same or different distinct H′ sequences and/or one, two, three, four, five, six, seven, eight, nine, ten, or more of the same or different distinct L′ sequences. The inclusion of one to ten (or more) additional H and L sequences, e.g., H″, H′″, L″, and L′″, are also encompassed by the presently disclosed subject matter.

Exemplary embodiments include, but are not limited, to sequences comprising any combination of various distinct H and various distinct L sequence such as: HL; LH; H′L; LH′; H′L′; L′H′; HLL; HHL; LLH; LHH; H′LL; H′HL; L′HH; L′LH; H′H′L; LH′H′; HLLL; LLLH; HLHL; LHLH; H′LLL; L′LLH; H′LHL; L′HLH; etc.

In certain embodiments, the present disclosure is directed to a method of expressing a SOI, comprising: providing a plurality of TI host cells; contacting the plurality of TI host cells with a plurality of vectors comprising one or more SOIs and at least one marker; introducing the one or more SOIs into one or more of the plurality of the TI host cells; selecting for TI cells expressing the sequence of interest; and culturing the cell under conditions suitable for expressing the sequence of interest and recovering the sequence of interest therefrom.

In certain embodiments the transfected host cell will comprise one or more sequence of interest where the sequence of interest comprises the sequence of one or more subunits of a multi-subunit protein complex. In certain embodiments, such polypeptide sequences can comprise fragments of such subunit sequences. In certain embodiments, the sequences of interest can comprise combinations of such subunit sequences. For example, but not by way of limitation, such combinations can comprise one, two, three, four, five, six, seven, eight, nine, ten, or more of a first subunit sequence and/or one, two, three, four, five, six, seven, eight, nine, ten, or more sequences of a second, third, fourth, fifth, sixth, seventh, eighth, ninth, tenth, or more subunit sequence. Moreover, in certain embodiments, such combinations can comprise one, two, three, four, five, six, seven, eight, nine, ten, or more of the same or different distinct variations of a first subunit sequence and/or one, two, three, four, five, six, seven, eight, nine, ten, or more of the same or different distinct variations of a second, third, fourth, fifth, sixth, seventh, eighth, ninth, tenth, or more subunit sequence.

In certain embodiments the transfected host cell will comprise one or more sequence of interest where the sequences of interest can comprise combinations of H and L sequences. For example, but not by way of limitation, such combinations can comprise one, two, three, four, five, six, seven, eight, nine, ten, or more H sequences and/or one, two, three, four, five, six, seven, eight, nine, ten, or more L sequences. Moreover, in certain embodiments, such combinations can comprise one, two, three, four, five, six, seven, eight, nine, ten, or more distinct H′ sequences and/or one, two, three, four, five, six, seven, eight, nine, ten, or more distinct L′ sequences. The inclusion of one to ten (or more) additional H and L sequences, e.g., H″, H′″, L″, and L′″, are also encompassed by the presently disclosed subject matter. Exemplary embodiments include, but are not limited, to sequences comprising any combination of various distinct H and various distinct L sequence such as: HL; LH; H′L; LH′; H′L′; L′H′; HLL; HHL; LLH; LHH; H′LL; H′HL; L′HH; L′LH; H′H′L; LH′H′; HLLL; LLLH; HLHL; LHLH; H′LLL; L′LLH; H′LHL; L′HLH; etc.

### 4.2 Regulated Systems

The presently disclosed subject matter also relates to regulated systems for use in multi-vector RMCE for the generation of TI host cells. For example, there are many cases where protein expression levels are not optimal mainly because the encoded proteins are difficult-to-express. The low expression level of difficult-to-express proteins can have diverse and difficult to identify causes. One possibility is the toxicity of the expressed proteins in the host cells. In such cases, a regulated expression system can be used to express toxic proteins where the sequences of interest encoding the proteins are under the control of an inducible promoter. In these systems, expression of the difficult-to-express proteins is only prompted when a regulator, e.g., small molecule, such as, but not limited to, tetracycline or its analogue, doxycycline (DOX), is added to the culture. Regulating the expression of toxic proteins could alleviate the toxic effects, allowing the cultures to achieve the desired cell growth prior to production. In certain embodiments, a TI expression system prepared according to the present disclosure comprises a SOI that is integrated into a specific locus, e.g., an exogenous nucleic acid sequence comprising one or more RRSs, and is transcribed under a regulated promoter operably linked thereto. In certain embodiments, a TI expression system prepared according to the present disclosure can be used to determine the underlying causes of low protein expression for a difficult-to-express molecule, such as, but not limited to, an antibody. In certain embodiments, the ability to selectively turn off the expression of a SOI in a TI system can be used to link expression of a SOI to an observed adverse effect.

In certain embodiments, to minimize transcriptional and cell line variability effects during the root cause analysis of difficult-to-express molecules, TI system can be used. For example, but not by way of limitation, the expression of the SOI in a TI host can be triggered by addition to the culture of a regulator, e.g., doxycycline. In certain embodiments, the TI vector utilizes a tetracycline-regulated promoter to express the SOI, which can be integrated into, e.g., an exogenous nucleic acid sequence comprising an RRS, which is itself integrated into an integration site in the host cell's genome, allowing for regulated expression of the SOI.

In certain embodiments, the TI system described in the present disclosure can be used to successfully determine the underlying cause(s) of low protein expression of an SOI, e.g., a therapeutic antibody, as compared to control cell line. In certain embodiments, once the lower relative expression of a SOI, e.g., a therapeutic antibody, in a TI cell line is confirmed, the intracellular accumulation and secretion levels of the SOI can be evaluated by leveraging protein translation inhibitor treatments, e.g., Dox and cycloheximide.

For example, but not by way limitation, such regulation can be based on gene switches for blocking or activating mRNA synthesis by regulated coupling of transcriptional repressors or activators to constitutive or minimal promoters. In certain non-limiting embodiments, repression can be achieved by binding the repressor proteins, e.g., where the proteins sterically block transcriptional initiation, or by actively repressing transcription through transcriptional silencers. In certain non-limiting embodiments, activation of mammalian or viral enhancerless minimal promoters can be achieved by the regulated coupling to an activation domain.

In certain embodiments, the conditional coupling of transcriptional repressors or activators can be achieved by using allosteric proteins that bind the promoters in response to external stimuli. In certain embodiments, the conditional coupling of transcriptional repressors or activators can be achieved by using intracellular receptors that are released from sequestering proteins and, thus, can bind target promoters. In certain embodiments, the conditional coupling of transcriptional repressors or activators can be achieved by using chemically induced dimerizers.

In certain embodiments, the allosteric proteins used in the TI systems of the present disclosure can be proteins that modulate transcriptional activity in response to antibiotics, bacterial quorum-sensing messengers, catabolites, or to the cultivation parameters, such as temperature, e.g. cold or heat. In certain embodiments, such TI systems can be catabolite-based, e.g., where a bacterial repressor that controls catabolic genes for alternative carbon sources has been transferred to mammalian cells. In certain embodiments, the repression of the target promoter can be achieved by cumate-responsive binding of the repressor CymR. In certain embodiments, the catabolite-based system can rely on the activation of chimeric promoters by 6-hydroxynicotine-responsive binding of the prokaryotic repressor HdnoR, fused to the Herpes simplex VP16 transactivation domain.

In certain embodiments the TI system can be a quorum-sensing-based expression system originated from prokaryotes that manage intra- and inter-population communication by quorum-sensing molecules. These quorum-sensing molecules bind to receptors in target cells, modulate the receptors' affinity to cognate promoters leading to the initiation of specific regulon switches. In certain embodiments, the quorum-sensing molecule can be the N-(3-oxo-octanoyl)-homoserine lactone in the presence of which, the TraR-p65 fusion protein activates expression from a minimal promoter fused to the TraR-specific operator sequence. In certain embodiments, the quorum-sensing molecule can be the butyrolactone SCB1 (racemic 2-(1′-hydroxy-6-methylheptyl)-3-(hydroxymethyl)-butanolide) in a system based on the Streptomyces coelicolor A3(2) ScbR repressor that binds its cognate operator OScbR in the absence of the SCB1. In certain embodiments, the quorum-sensing molecule can be homoserine-derived inducers used in a TI system wherein Pseudomonas aeruginosa quorum-sensing repressors RhlR and LasR are fused to the SV40 T-antigen nuclear localization sequence and the Herpes simplex VP16 domain and can activate promoters containing specific operator sequences (las boxes).

In certain embodiments, the inducing molecules that modulate the allosteric proteins used in the TI systems of the present disclosure can be, but are not limited to, cumate, isopropyl-β-D-galactopyranoside (IPTG), macrolides, 6-hydroxynicotine, doxycycline, streptogramins, NADH, tetracycline.

In certain embodiments, the intracellular receptors used in the TI systems of the present disclosure can be cytoplasmic or nuclear receptors. In certain embodiments, the TI systems of the present disclosure can utilize the release of transcription factors from sequestering and inhibiting proteins by using small molecules. In certain embodiments, the TI systems of the present disclosure can rely on steroid-regulation, wherein a hormone receptor is fused to a natural or an artificial transcription factor that can be released from HSP90 in the cytosol, migrate into the nucleus and activate selected promoters. In certain embodiments, mutant receptors can be used that are regulated by synthetic steroid analogs in order to avoid crosstalk by endogenous steroid hormones. In certain embodiments the receptors can be an estrogen receptor variant responsive to 4-hydroxytamoxifen or a progesterone-receptor mutant inducible by RU486. In certain embodiments, the nuclear receptor-derived rosiglitazone-responsive transcription switch based on the human nuclear peroxisome proliferator-activated receptor γ(PPARγ) can be used in the TI systems of the present disclosure. In certain embodiments, a variant of steroid-responsive receptors can be the RheoSwitch, that is based on a modified Choristoneura fumiferana ecdysone receptor and the mouse retinoid X receptor (RXR) fused to the Gal4 DNA binding domain and the VP16 trans-activator. In the presence of synthetic ecdysone, the RheoSwitch variant can bind and activate a minimal promoter fused to several repeats of the Gal4-response element.

In certain embodiments, the TI systems disclosed herein can utilize chemically induced dimerization of a DNA-binding protein and a transcriptional activator for the activation of a minimal core promoter fused with a cognate operator. In certain embodiments, the TI systems disclosed herein can utilize the rapamycin-regulated dimerization of FKBP with FRB. In this system the FRB is fused to the p65 trans-activator and FKBP is fused to a zinc finger domain specific for cognate operator sites placed upstream of an engineered minimal interleukin-12 promoter. In certain embodiments, the FKBP can be mutated. In certain embodiments, the TI systems disclosed herein can utilize bacterial gyrase B subunit (GyrB), where GyrB dimerizes in the presence of the antibiotic coumermycin and dissociates with novobiocin.

In certain embodiments, the TI systems of the present disclosure can be used for regulated siRNA expression. In certain embodiments, the regulated siRNA expression system can be a tetracycline, a macrolide, or an OFF- and ON-type QuoRex system. In certain embodiments, the TI system can utilize a Xenopus terminal oligopyrimidine element (TOP), which blocks translational initiation by forming hairpin structures in the 5′ untranslated region.

In certain embodiments, the TI systems described in the present disclosure can utilize gas-phase controlled expression, e.g., acetaldehyde-induced regulation (AIR) system. The AIR system can employ the Aspergillus nidulans AlcR transcription factor, which specifically activates the PAIR promoter assembled from AlcR-specific operators fused to the minimal human cytomegalovirus promoter in the presence of gaseous or liquid acetaldehyde at nontoxic concentrations.

In certain embodiments, the TI systems of the present disclosure can utilize a Tet-On or a Tet-Off system. In such systems, expression of a one or more SOIs can be regulated by tetracycline or its analogue, doxycycline.

In certain embodiments, the TI system of the present disclosure can utilize a PIP-on or a PIP-off system. In such systems, the expression of SOIs can be regulated by, e.g., pristinamycin, tetracycline and/or erythromycin.

### 5. Products

The host cells of the present disclosure can be used for the expression of any molecule of interest. In certain embodiments, the host cells of the present disclosure can be used for the expression of polypeptides, e.g., mammalian polypeptides. Non-limiting examples of such polypeptides include hormones, receptors, fusion proteins, regulatory factors, growth factors, complement system factors, enzymes, clotting factors, anti-clotting factors, kinases, cytokines, CD proteins, interleukins, therapeutic proteins, diagnostic proteins and antibodies. In certain embodiments, the host cells of the present disclosure can be used for the expression of chaperones, protein modifying enzymes, shRNA, gRNA or other proteins or peptides while expressing a therapeutic protein or molecule of interest constitutively or regulated.

In certain embodiments, the polypeptide of interest is a bi-specific, tri-specific or multi-specific polypeptide, e.g. a bi-specific antibody.

The host cells of the present disclosure can be employed in the production of large quantities of a molecule of interest in a shorter timeframe as compared to cells, e.g., non-TI cells, used in conventional cell culture methods. In certain embodiments, the host cells of the present disclosure can be employed for improved quality of the molecule of interest as compared to cells, e.g., non-TI cells, used in conventional cell culture methods. In certain embodiments, the host cells of the present disclosure can be used to enhance seed train stability by preventing chronic toxicity that can be caused by products that can cause cell stress and clonal instability over time. In certain embodiments, the host cells of the present disclosure can be used for the optimal expression of acutely toxic products.

In certain embodiments, the host cells and systems of the present disclosure can be used for cell culture process optimization and/or process development.

In certain embodiments, the host cells of the present disclosure can be used for the constitutive expression of selected subunits of a therapeutic molecule and the regulated expression of other, different subunits of the same therapeutic molecule. In certain embodiments the therapeutic molecule can be a fusion protein. In certain embodiments, the host cells of the present disclosure can be used to understand the roles and effects of each antibody subunit in the expression and secretion of fully assembled antibody molecules.

In certain embodiments, the host cells of the present disclosure can be used as an investigational tool. In certain embodiments, the host cells of the present disclosure can be used as a diagnostic tool to map out the root causes of low protein expression for problematic molecules in various cells. In certain embodiments, the host cells of the present disclosure can be used to directly link an observed phenomenon or cellular behavior to the transgene expression in the cells. The host cell of the present disclosure can also be used to demonstrate whether or not an observed behavior is reversible in the cells. In certain embodiments, the host cells of the present disclosure can be exploited to identify and mitigate problems with respect to transgene(s) transcription and expression in cells.

In certain embodiments, the host cells of the present disclosure can be used for swapping transgene subunits, such as but not limited to, HC and LC subunits of an antibody, of a difficult-to-express molecule with that of an average molecule in the system to identify the problematic subunit(s). In certain embodiments, amino acid sequence analysis can then be used to narrow down and focus on the amino acid residues or regions that might be responsible for low protein expression.

### EXAMPLES

### Example 1: Comparison of Two Vector RMCE to Multi-Vector RMCE

In the instant example, differences in various CLD parameters are identified when multi-vector RMCE (in this example, three vector RMCE) is employed as compared to two vector RMCE.

### Plasmid Construction

For two-vector based RMCE, front and back antibody targeting vectors were constructed. The HC and LC cDNAs of an antibody were cloned into the front vector backbone containing the L3, a promoter and a start codon (ATG) followed by the LoxFAS sequence, and the back vector backbone containing the LoxFAS, Pac lacking a start codon and the 2L sequence. The Cre recombinase plasmid (pOG231) was used for all RMCE processes.

For three-vector based RMCE, three antibody targeting vectors (plasmid 1, 2 and 3) were constructed. Plasmid 1 is identical to the front vector in two-vector based RMCE described above. Plasmid 2 backbone contains the LoxFAS, Pac lacking a start codon, a CMV promoter and the Frt3 sequence. Plasmid 3 backbone contains the Frt3, NeoR and the Frt sequence. The coding sequence of FlpO recombinase was synthesized based on mouse codon-optimized sequence. The sequence was cloned into a proprietary expression vector driven by CMV promoter and containing a polyadenylation sequence.

### Transfection and RMCE

For two-vector based RMCE, front and back antibody targeting vectors containing the HC and LC, and pOG231 are transfected into TI host cells by electroporation using the Maxcyte STX (Maxcyte, Gaithersburg, MD).

For three-vector based RMCE, plasmid 1, 2, 3 antibody targeting vectors containing the HC and LC, and pOG231 and FlpO expression plasmid are transfected into TI host cells by electroporation using the Maxcyte STX (Maxcyte, Gaithersburg, MD).

48 hours after transfection, the cells were pelleted by centrifugation. The pellet was then resuspended in proprietary DMEM/F12-based media containing selection and cultured at 37° C. and 5% CO₂ until transfection pools recovered.

### Fed-Batch Shake Flask Evaluation of the RMCE Pool

Fed-batch production cultures were performed in shake flasks with proprietary chemically defined media together with bolus feeds on day 1, 3, and 4. Temperature was maintained at 35° C. throughout the run. Day 7 titers were determined using protein A affinity chromatography with UV detection. Percent viability and viable cell count was determined using the Vi-Cell XR instrument (Beckman Coulter).

Copy Number Analysis by Digital Droplet PCR (ddPCR)

Genomic DNA from 1×10⁶ cell pellets was purified by the MagNA Pure 96 instrument (Roche, Molecular Systems) using the MagNA Pure 96 DNA and Viral NA Small Volume Kit (Roche Molecular Systems, Cat. No. 06543588001). The copy numbers of the Chinese hamster Bel-2-associated X protein (Bax) and Albumin (Alb) were used as reference for the determination of gene copy of HC and LC respectively. ddPCR reactions were prepared following the manual of QX200 ddPCR system (Bio-Rad, Cat. No. 186-3010). In brief, ddPCR reactions for were set up with HC or LC probes labeled with HEX, and Bax or Alb probes labeled with FAM in 20 dl per reaction. Samples were then placed into a QX200 droplet generator, and droplets were transferred into 96-well plates for PCR in a thermal cycler followed by droplet reading. The cycling conditions are as follows: 10 min at 95° C., 40 cycles of 30 s at 94° C., and 1 min at 60° C. followed by 10 min at 98° C. for enzyme deactivation. The primers and probes used in this study were designed using primer express v3.0 (Life Technologies).

mRNA Expression by Digital Droplet Reverse Transcriptase PCR

Total RNA from 1×10⁶ cell pellets was purified by the MagNA Pure 96 instrument (Roche, Molecular Systems) using the MagNA Pure 96 Cellular RNA Large Volume Kit (Roche Molecular Systems, Cat. No. 05467535001) and quantified using a Nanodrop 2000 Spectrophotometer (Thermo Fisher Scientific, Cat. No. ND-2000). Approximately 20-40 μg of RNA was used to perform ddRT-PCR using the One-Step RT-ddPCR Advanced Kit for Probes (Bio-Rad, Cat. No. 1864022). Primers and probes for IgG heavy chain and light chain were identical to those used for gene copy analysis. Droplet generation, thermal cycling, and droplet reading were executed using the Bio-Rad QX200 ddPCR system (Bio-Rad, Cat. No. 186-3010). Thermal cycling conditions were 50° C. for 1 hr, 95° C. for 10 min, then 40 cycles of 95° C. for 30 sec and 58° C. for 1 min, followed by 98° C. for 10 min for enzyme deactivation. The resulting concentrations were normalized to copies per 20 μg of total RNA.

TABLE 2
Differences in various CLD parameters are identified when multi-vector RMCE (in
this example, three vector RMCE) is employed as compared to two vector RMCE.

| | Pool Titer | Pool Qp | Pool IVCC | Copy#/Cell | | mRNA/Cell | |
|---|---|---|---|---|---|---|---|
| Plasmids | (g/L) | (pg/cell/day) | (10\hat{ }8 cell-d/L) | HC | LC | HC | LC |
| 2 | 6.2 | 18.8 | 3309 | 2 | 4 | 6565 | 19919 |
| 3 | 7.8 | 23.8 | 3282 | 3.3 | 6.6 | 8439 | 38899 |

### Example 2: Ambr© 250 Comparison of Two Vector RMCE to Multi-Vector RMCE

In the instant example, gene copy number and mRNA expression are compared between cell lines expressing an antibody cultured in an Ambr® 250 system (TAP Biosystem), but where one cell line is prepared using a two-plasmid RMCE approach and the second cell line is prepared using a multi-vector RMCE approach. In the two-plasmid approach, each plasmid comprises one copy of the heavy chain coding sequence and two copies of the light chain coding sequence (“HLL-HLL”). In the multi-vector approach, each plasmid similarly comprises one copy of the heavy chain coding sequence and two copies of the light chain coding sequence. Given the presence of a third plasmid in the multi-vector RMCE, however, the approach results in an “HLL-HLL-HLL” configuration.

Twelve clones are evaluated in the Ambr® 250 system (TAP Biosystem). The Ambr® 250 system is a high throughput automated approach employing single-use 250 mL mini bioreactors, which are used in this example as recommended by the manufacturer. Briefly, thirty million cells are seeded into proprietary chemically defined production medium having a pH of 7.15, and the culture temperature is maintained at 37° C. for the first 48 hours. The culture temperature is then shifted to 35° C. for the remainder of the culture. The 14-day fed-batch culture employs five bolus feeds on days 1, 3, 6, 9, and 12. Cell viability and viable cell counts are monitored by trypan blue dye exclusion using a BioProfile Flex2 (Nova Biomedical).

### Example 3: Comparison of Two Vector RMCE to Two-Site Multi-Vector RMCE

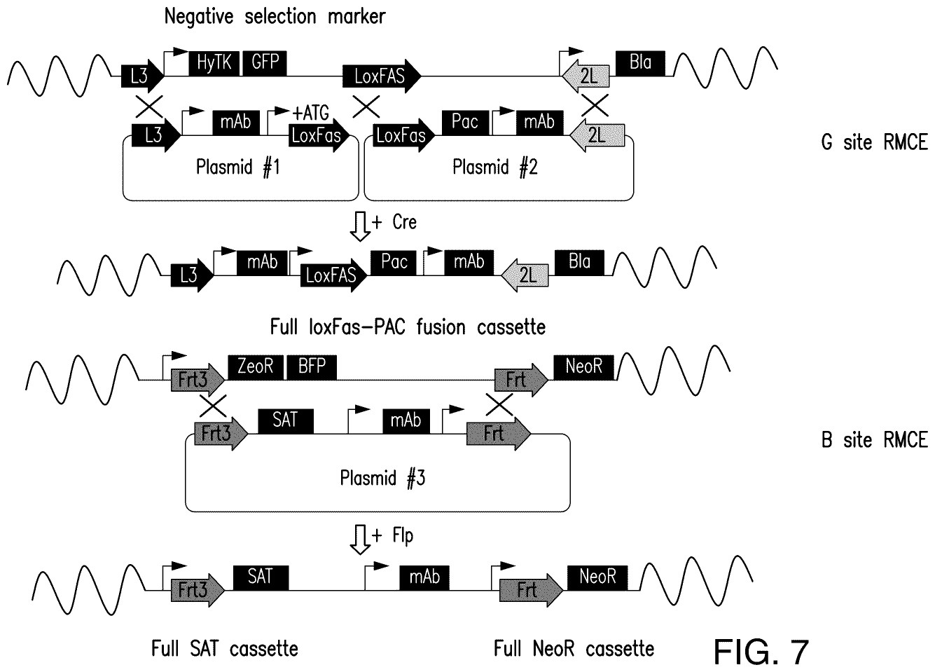

In the instant example, differences in various CLD parameters are identified when multi-vector RMCE (in this example, three vector RMCE, where two vectors are integrated at a first site and a third vector is integrated at a second site) is employed as compared to two vector RMCE. Antibody expressing constructs were prepared as outlined in Example 1, except as illustrated in FIG. 7. Briefly, while green fluorescent protein (GFP) landing pad at the “G” site is used as illustrated for the incorporation of the first two plasmids as described above and in the associated figures, the blue fluorescent protein (BFP) landing pad at the “B” site consists of a zeocin resistance gene (ZeoR) and a (BFP) flanked by Frt and Frt3 recombinase site. A promoterless Neo is placed downstream of the Frt site. The antibody targeting vector for “B” site contains an SAT gene and the mAb expression cassette that encodes various copies of HC and LC cDNA, followed by a promoter, all flanked by Frt3 and Frt.

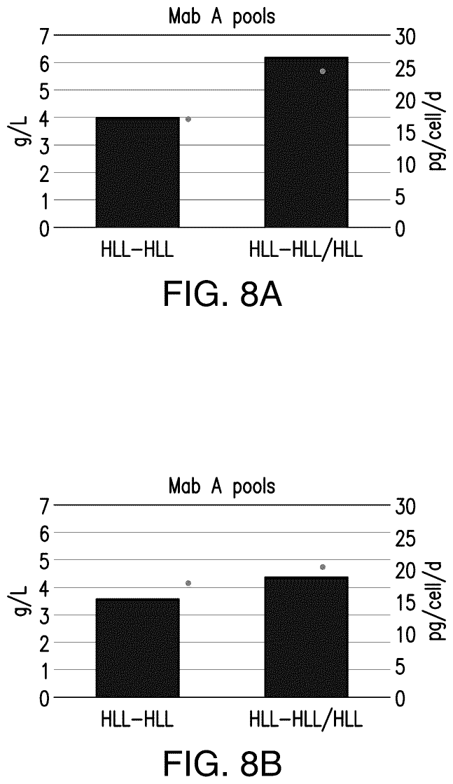

FIGS. 8A-8B depict a comparison of the productivities of TI transfection pools using two-plasmid based RMCEs and 2-site multi-vector plasmid (in this case, three plasmid) based RMCEs. Titer (bars) and Qp (dots) are both higher for the 2-site multi-vector plasmid-based TI pools of mAb A (FIG. 8A) and TI pools of mAb B (FIG. 8b).

### Example 4: Comparison of Two Vector RMCE to Multi-Vector RMCE

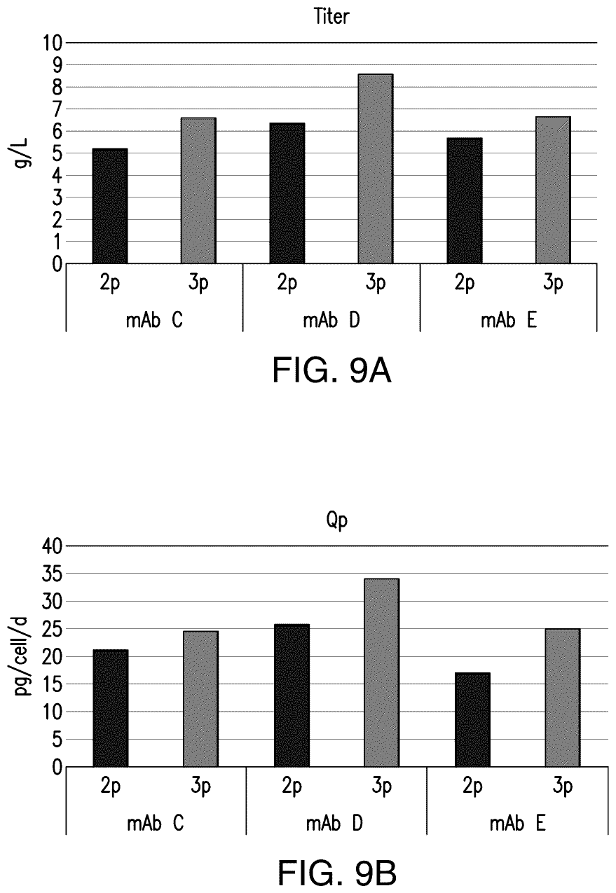

In this example, two vector and multi-vector RMCE (in this case three plasmid RMCE) shake flask cultures of mAb C, mAb D, and mAb E were prepared as outlined above and cultured for seven days. Upon completion of the seven day cultures, certain CLD parameters were assessed for the various TI pools of mAb C, mAb D, and mAb E as reported in Table 3. These results are also graphically depicted in FIG. 9A (titer) and FIG. 9B (Qp).

TABLE 3

| Molecule | | | | | | |
|---|---|---|---|---|---|---|
| (7-day shake | | Titer | Qp | | mRNA HC | mRNA LC |
| flask) | Plasmids | (g/L) | (pcd) | IVCC | (copy/cell) | (copy/cell) |
| mAb C | HLL-HLL | 5.2 | 21.27 | 2454.60 | 11400 | 32282 |
| | HLL-HLL-HLH | 6.7 | 24.63 | 2704.50 | 16697 (+45%) | 44598 (+38%) |
| mAb D | HLL-HLL | 6.4 | 25.97 | 2461.00 | 10691 | 44752 |
| | HLL-HLL-HLH | 8.6 | 34.18 | 2501.60 | 15289 (+43%) | 59556 (+33%) |
| mAb E | HLL-HLL | 4.7 | 13.8 | 3405 | | |
| | HLL-HLL-HLH | 5.3 | 20.9 | 2523 | | |

### Example 5: Ambr15© Comparison of Two Vector RMCE to Multi-Vector RM

In this example, cell lines expressing mAb C or mAb D were prepared using either two vector or multi-vector RMCE (in this case, three plasmid RMCE) as outlined above. Ambr15© cultures (TAP Biosystem) of individual clones each of mAb C and mAb D (four each of two vector RMCE and multi-vector RMCE for each antibody) were performed and the multi-vector RMCE clones exhibited higher average titer and higher average Qp relative to two vector RMCE clones as reported in Table 4.

TABLE 4

| | | average | Average |
|---|---|---|---|
| Molecule | | Titer | Qp |
| (Ambr15) | Plasmids | (g/L) | (pcd) |
| mAb C | HLL-HLL | 8.7 ± 0.25 | 29.1 ± 3.5 |
| | HLL-HLL-HLH | 10.0 ± 0.82 | 37.9 ± 6.8 |
| mAb D | HLL-HLL | 9.6 ± 4.1 | 18.6 ± 8.7 |
| | HLL-HLL-HLH | 10.9 ± 2.7 | 24.6 ± 7.1 |

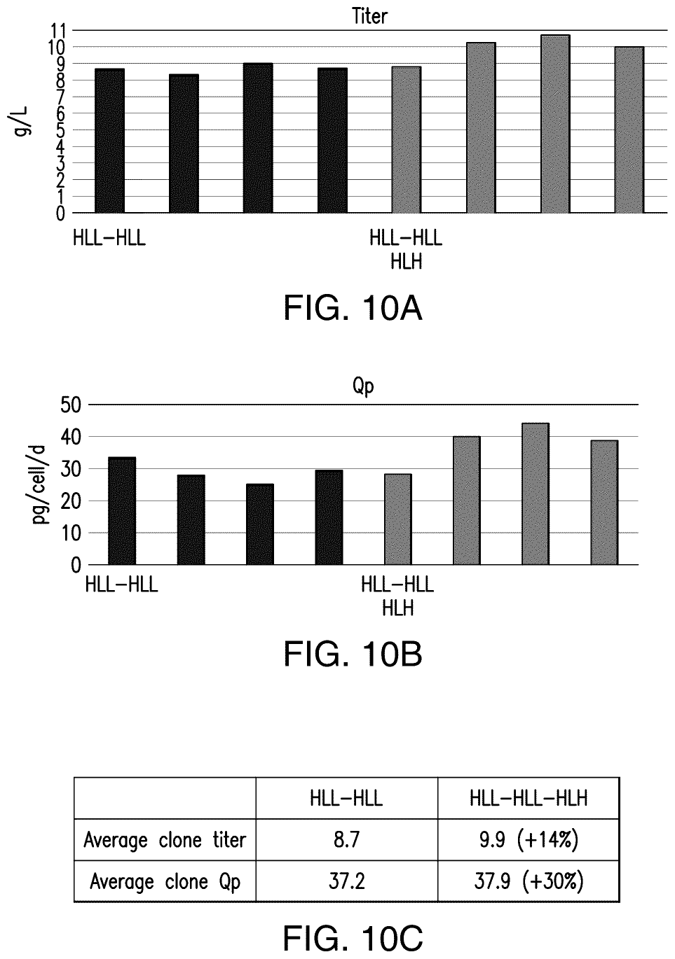

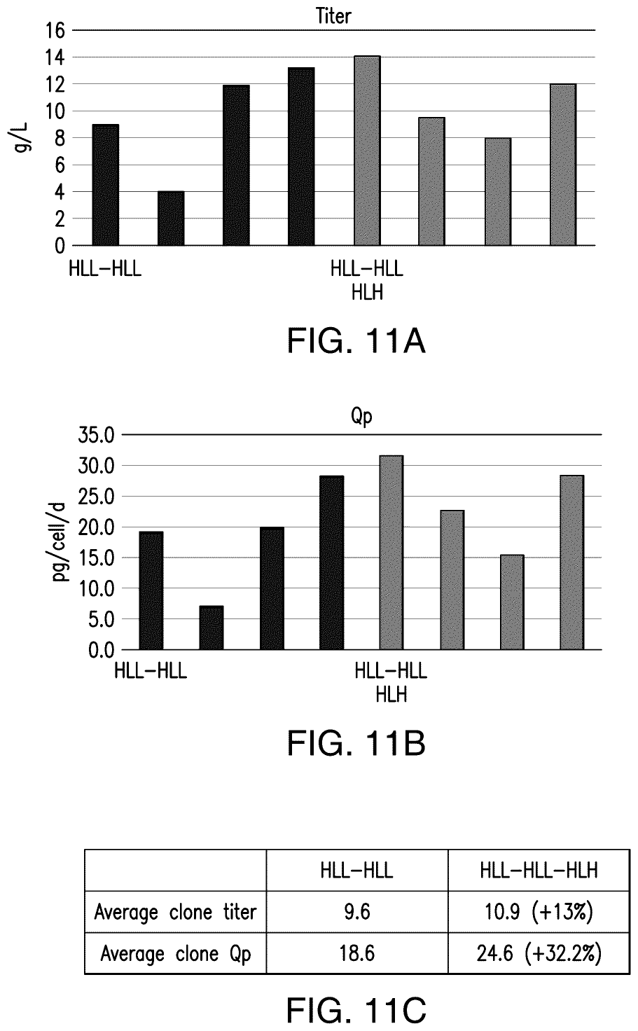

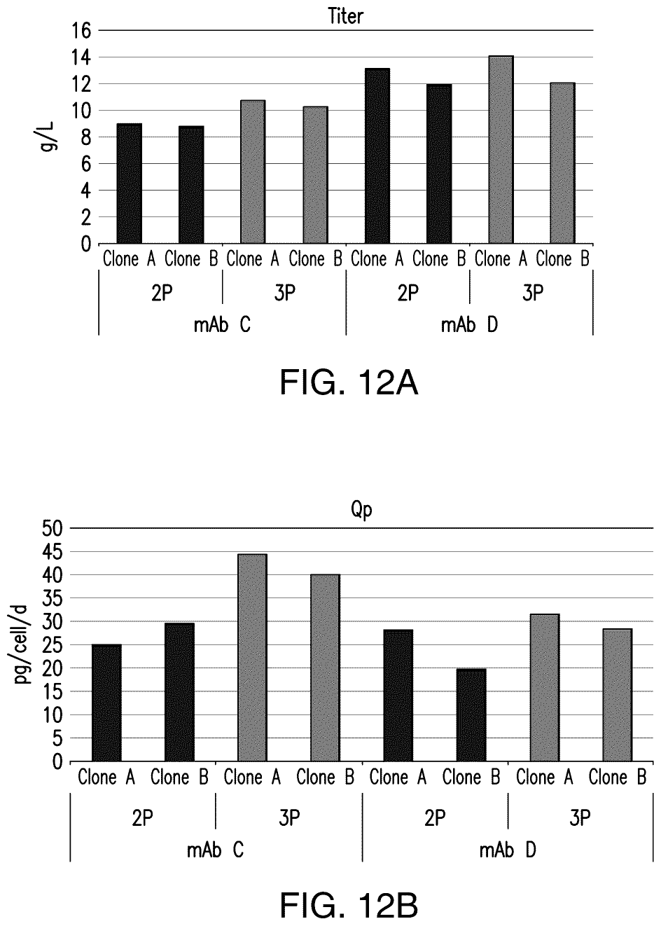

FIG. 10A graphically illustrates the titer observed for each of the eight mAb C clones while FIG. 10B graphically illustrates the Qp observed for each of those mAb C eight clones. FIG. 10C compares the average mAb C clone titers and mAb C clone Qp. Similarly, FIG. 11A graphically illustrates the titer observed for each of the eight mAb D clones while FIG. 11B graphically illustrates the Qp observed for each of those eight mAb D clones. FIG. 11C compares the average mAb D clone titers and average mAb D clone Qp. In contrast, FIGS. 12A-12B compares titer (FIG. 12A) and Qp (FIG. 12B) for two specific clones (Clone A and Clone B) for each of the two vector and multi-vector approaches. The data graphically presented in FIGS. 12A-12B is also presented in Table 5, below.

TABLE 5

| | HLL-HLL | HLL-HLL-HLH |
|---|---|---|
| mAb C | | |
| Clone A titer | 9.0 | 10.8 |
| (Qp) | (25.2) | (44.4) |
| Clone B titer | 8.8 | 10.3 |
| (Qp) | (29.6) | (40) |
| mAb D | | |
| Clone A titer | 13.2 | 14.1 |
| (Qp) | (28.3) | (31.7) |
| Clone B titer | 11.9 | 12.1 |
| (Qp) | (19.8) | (28.4) |

The preceding examples are merely illustrative of the presently disclosed subject matter and should not be considered as limiting in any way.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the compositions and methods of the disclosed subject matter without departing from the spirit or scope of the disclosed subject matter. Thus, it is intended that the disclosed subject matter include modifications and variations that are within the scope of the appended claims and their equivalents.

Various publications, patents and patent applications are cited herein, the contents of which are hereby incorporated by reference in their entireties.

What is claimed is:

1. A targeted integration (TI) host cell comprising an exogenous nucleotide sequence integrated at an integration site that is within a sequence:
a) at least about 90% homologous to all or part of nucleotides 41190-45269 of NW_006874047.1, all or part of nucleotides 63590-207911 of NW_006884592.1, all or part of nucleotides 253831-491909 of NW_006881296.1, all or part of nucleotides 69303-79768 of NW_003616412.1, all or part of nucleotides 293481-315265 of NW_003615063.1, all or part of nucleotides 2650443-2662054 of NW_006882936.1, or all or part of nucleotides 82214-97705 of NW_003615411.1; or
b) at least about 90% homologous to all or part of nucleotides 45270-45490 of NW_006874047.1, all or part of nucleotides 207912-792374 of NW_006884592.1, all or part of nucleotides 491910-667813 of NW_006881296.1, all or part of nucleotides 79769-100059 of NW_003616412.1, all or part of nucleotides 315266-362442 of NW_003615063.1, all or part of nucleotides 2662055-2701768 of NW_006882936.1, or all or part of nucleotides 97706-105117 of NW_003615411.1; and
wherein the exogenous nucleotide sequence comprises four or more incompatible recombination recognition sequences (RRSs).

2. The TI host cell of claim 1, wherein the RRSs are selected from the group consisting of a LoxP sequence, a LoxP L3 sequence, a LoxP 2L sequence, a LoxFas sequence, a Lox5 11 sequence, a Lox2272 sequence, a Lox2372 sequence, a Lox5 171 sequence, a Loxm2 sequence, a Lox71 sequence, a Lox66 sequence, a FRT sequence, a Bxbl attP sequence, a Bxbl attB sequence, a φC31 attP sequence, and a φC31 attB sequence.

3. The TI host cell of claim 1, wherein the RRSs are recognized by recombinases selected from the group consisting of Cre recombinase, FLP recombinase, Bxbl integrase, and a φC31 integrase.

4. The TI host cell of claim 1, wherein the exogenous nucleotide sequence comprises a selection marker located between the 5′-most RRS and the next RRS in the 3′ direction.

5. The TI host cell of claim 4, wherein the selection marker is selected from the group consisting of aminoglycoside phosphotransferase (APH), hygromycin phosphotransferase (HYG), neomycin, G418 APH), dihydrofolate reductase (DHFR), thymidine kinase (TK), glutamine synthetase (GS), asparagine synthetase, tryptophan synthetase (indole), histidinol dehydrogenase (histidinol D), blasticidin, bleomycin, phleomycin, chloramphenicol, Zeocin, and mycophenolic acid.

6. The TI host cell of claim 5, further comprising a second selection marker, wherein the first and the second selection markers are different.

7. The TI host cell of claim 6, wherein the second selection marker is selected from the group consisting of aminoglycoside phosphotransferase (APH), hygromycin phosphotransferase (HYG), neomycin, G418 APH), dihydrofolate reductase (DHFR), thymidine kinase (TK), glutamine synthetase (GS), asparagine synthetase, tryptophan synthetase (indole), histidinol dehydrogenase (histidinol D), blasticidin, bleomycin, phleomycin, chloramphenicol, Zeocin, and mycophenolic acid.

8. The TI host cell of claim 6, further comprising a third selection marker and an internal ribosome entry site (IRES), wherein the IRES is operably linked to a third selection marker.

9. The TI host cell of claim 8, wherein the third selection marker is different from the first or the second selection marker.

10. The TI host cell of claim 9, wherein the third selection marker is selected from the group consisting of a green fluorescent protein (GFP) marker, an enhanced GFP (eGFP) marker, a synthetic GFP marker, a yellow fluorescent protein (YFP) marker, an enhanced YFP (eYFP) marker, a cyan fluorescent protein (CFP) marker, a mPlum marker, a mCherry marker, a tdTomato marker, a mStrawberry marker, a J-red marker, a DsRed-monomer marker, a mOrange marker, a mKO marker, a mCitrine marker, a Venus marker, a YPet marker, an Emerald6 marker, a CyPet marker, a mCFPm marker, a Cerulean marker, and a T-Sapphire marker.

11. The TI host cell of claim 1, wherein the TI host cell is a mammalian host cell.

12. The TI host cell of claim 11, wherein the TI host cell is a hamster host cell, a human host cell, a rat host cell, or a mouse host cell.

13. The TI host cell of claim 11, wherein the TI host cell is a Chinese hamster ovary (CHO) host cell, a CHO K1 host cell, a CHO K1 SV host cell, a DG44 host cell, a DUKXB-1 host cell, a CHOK1 S host cell, or a CHO K1M host cell.

14. A method of preparing a TI host cell expressing sequences of interest (SOIs) comprising:
a) providing a TI host cell comprising an exogenous nucleotide sequence integrated at an integration site that is within a sequence:
a. at least about 90% homologous to all or part of nucleotides 41190-45269 of NW_006874047.1, all or part of nucleotides 63590-207911 of NW_006884592.1, all or part of nucleotides 253831-491909 of NW_006881296.1, all or part of nucleotides 69303-79768 of NW_003616412.1, all or part of nucleotides 293481-315265 of NW_003615063.1, all or part of nucleotides 2650443-2662054 of NW_006882936.1, or all or part of nucleotides 82214-97705 of NW_003615411.1; or
b. at least about 90% homologous to all or part of nucleotides 45270-45490 of NW_006874047.1, all or part of nucleotides 207912-792374 of NW_006884592.1, all or part of nucleotides 491910-667813 of NW_006881296.1, all or part of nucleotides 79769-100059 of NW_003616412.1, all or part of nucleotides 315266-362442 of NW_003615063.1, all or part of nucleotides 2662055-2701768 of NW_006882936.1, or all or part of nucleotides 97706-105117 of NW_003615411.1; and
wherein the exogenous nucleotide sequence comprises four or more incompatible RRSs;
b) introducing into the cell provided in a) at least three vectors, each vector comprising:
a. two RRSs matching two sequentially oriented RRSs on the integrated exogenous nucleotide sequence; and
b. each pair of RRSs flanking at least one exogenous SOI and at least one second selection marker;
c) introducing recombinases or nucleic acids encoding recombinases, wherein the recombinases recognize the RRSs; and
d) selecting for TI cells expressing the selection markers to thereby isolate a TI host cell expressing the SOIs.

15. The method of claim 14, wherein the recombinases are selected from Cre recombinase, FLP recombinase, Bxbl integrase, and a (φC31 integrase.

16. The method of claim 14, wherein the SOIs encode a single chain antibody, an antibody light chain, an antibody heavy chain, a single-chain Fv fragment (scFv), or an Fc fusion protein.

17. The method of claim 14, wherein the TI host cell is a mammalian host cell.

18. The method of claim 17, wherein the TI host cell is a CHO host cell, a CHO K1 host cell, a CHO K1SV host cell, a DG44 host cell, a DUKXB-11 host cell, a CHOK1S host cell, or a CHO K1M host cell.

19. The method of claim 14, wherein the vector is selected from the group consisting of an adenovirus vector, an adeno-associated virus vector, a lentivirus vector, a retrovirus vector, an integrating phage vector, a non-viral vector, a transposon and/or transposase vector, an integrase substrate, and a plasmid.

20. A method for expressing SOIs comprising:
a) providing a TI host cell comprising an exogenous nucleotide sequence integrated at an integration site that is within a sequence:
a. at least about 90% homologous to all or part of nucleotides 41190-45269 of NW_006874047.1, all or part of nucleotides 63590-207911 of NW_006884592.1, all or part of nucleotides 253831-491909 of NW_006881296.1, all or part of nucleotides 69303-79768 of NW_003616412.1, all or part of nucleotides 293481-315265 of NW_003615063.1, all or part of nucleotides 2650443-2662054 of NW_006882936.1, or all or part of nucleotides 82214-97705 of NW_003615411.1; or
b. at least about 90% homologous to all or part of nucleotides 45270-45490 of NW_006874047.1, all or part of nucleotides 207912-792374 of NW_006884592.1, all or part of nucleotides 491910-667813 of NW_006881296.1, all or part of nucleotides 79769-100059 of NW_003616412.1, all or part of nucleotides 315266-362442 of NW_003615063.1, all or part of nucleotides 2662055-2701768 of NW_006882936.1, or all or part of nucleotides 97706-105117 of NW_003615411.1; and
wherein the exogenous nucleotide sequence comprises four or more incompatible RRSs;
b) introducing into the cell provided in a) at least three vectors, each vector comprising:
a. two RRSs matching two sequentially oriented RRSs on the integrated exogenous nucleotide sequence; and
b. each pair of RRSs flanking at least one exogenous SOI and at least one second selection marker;
c) introducing recombinases or nucleic acids encoding recombinases, wherein the recombinases recognize the RRSs;
d) selecting for TI cells expressing the selection markers to thereby isolate a TI host cell expressing the SOIs; and
e) culturing the cell in d) under conditions suitable for expressing the SOIs and recovering the expressed protein therefrom.

21. The method of claim 20, wherein the recombinases are selected from Cre recombinase, FLP recombinase, Bxbl integrase, and a φC31 integrase.

22. The method of claim 20, wherein the SOIs encode a single chain antibody, an antibody light chain, an antibody heavy chain, a single-chain Fv fragment (scFv), or an Fc fusion protein.

23. The method of claim 20, wherein the TI host cell is a mammalian host cell.

24. The method of claim 20, wherein the vector is selected from the group consisting of an adenovirus vector, an adeno-associated virus vector, a lentivirus vector, a retrovirus vector, an integrating phage vector, a non-viral vector, a transposon and/or transposase vector, an integrase substrate, and a plasmid.

25. A method for producing a recombinant mammalian cell comprising a nucleic acid encoding an antibody, comprising:
a) providing a mammalian cell comprising at least a single exogenous nucleic acid incorporated at a predetermined locus of the genome of the mammalian cell comprising four or more incompatible RRSs;
b) introducing into the recombinant mammalian cell of a), at least three vectors, each vector comprising a pair of incompatible RRSs matching two of the incompatible RRSs comprised in the exogenous nucleic acid incorporated at a predetermined locus of the genome of the mammalian cell and each pair of incompatible RRSs flank one or more SOIs where the SOIs encode an antibody and/or one or more selection markers;
c) introducing one or more recombinases, simultaneously or sequentially, with the introduction of the at least three vectors comprising the SOIs and/or selection markers; and
d) selecting for cells expressing one or more of the SOIs and/or selection markers,
thereby producing a recombinant mammalian cell comprising nucleic acid SOIs encoding the antibody.

26. The method of claim 25, wherein the exogenous nucleic acid is incorporated at a locus:
a. at least about 90% homologous to all or part of nucleotides 41190-45269 of NW_006874047.1, all or part of nucleotides 63590-207911 of NW_006884592.1, all or part of nucleotides 253831-491909 of NW_006881296.1, all or part of nucleotides 69303-79768 of NW_003616412.1, all or part of nucleotides 293481-315265 of NW_003615063.1, all or part of nucleotides 2650443-2662054 of NW_006882936.1, or all or part of nucleotides 82214-97705 of NW_003615411.1; or
b. at least about 90% homologous to all or part of nucleotides 45270-45490 of NW_006874047.1, all or part of nucleotides 207912-792374 of NW_006884592.1, all or part of nucleotides 491910-667813 of NW_006881296.1, all or part of nucleotides 79769-100059 of NW_003616412.1, all or part of nucleotides 315266-362442 of NW_003615063.1, all or part of nucleotides 2662055-2701768 of NW_006882936.1, or all or part of nucleotides 97706-105117 of NW_003615411.1.

27. The method of claim 25, wherein the recombinases are selected from Cre recombinase, FLP recombinase, Bxbl integrase, and a φC31 integrase.

28. The method of claim 25, wherein the SOIs encode a single chain antibody, an antibody light chain, an antibody heavy chain, a single-chain Fv fragment (scFv), or an Fc fusion protein.

29. The method of claim 25, wherein the vectors are selected from the group consisting of adenovirus vectors, adeno-associated virus vectors, lentivirus vectors, retrovirus vectors, integrating phage vectors, non-viral vectors, transposon and/or transposase vectors, integrase substrates, and plasmids.

30. The method of claim 25, wherein the recombinant mammalian cells comprises at least two exogenous nucleic acids incorporated at predetermined loci of the genome of the mammalian cell comprising, each exogenous nucleic acid comprising four or more incompatible RRSs, where the exogenous nucleic acids can comprise the same or different RRSs.
